SEQUENCE LISTING (18 sequences)

SEQ ID NO: 1 - protein, 6 aa, Artificial sequence
  Synthetic sequence N-terminal amino acid tag
AGGCGH

SEQ ID NO: 2 - protein, 10 aa, Artificial sequence
  Synthetic sequence
LAAALEHHHH

SEQ ID NO: 3 - protein, 12 aa, Artificial sequence
  Synthetic sequence
LAAALEHHHHHH

SEQ ID NO: 4 - protein, 14 aa, Artificial sequence
  Synthetic sequence Linker
CKLAAALEHHHHHH

SEQ ID NO: 5 - protein, 21 aa, Artificial sequence
  Synthetic sequence General formula of linker sequence
CXXXXXXXXXXHHHHHHHHHH

SEQ ID NO: 6 - protein, 31 aa, Artificial sequence
  Synthetic sequence General formula of bioconjugate
XXXXXXXXXXCXXXXXXXXXXHHHHHHHHHH

SEQ ID NO: 7 - protein, 7 aa, Artificial sequence
  Synthetic sequence Linker
KLAAALE

SEQ ID NO: 8 - protein, 6 aa, Artificial sequence
  Synthetic sequence Tag
HHHHHH

SEQ ID NO: 9 - DNA, 33 bp, Artificial sequence
  Synthetic sequence Forward primer
cacacacatatggagaaactgggaaagctccaa

SEQ ID NO: 10 - DNA, 42 bp, Artificial sequence
  Synthetic sequence Reverse primer
cacacaaagctttcagcatttctcagcgctctggagatcgcg

SEQ ID NO: 11 - DNA, 39 bp, Artificial sequence
  Synthetic sequence Reverse primer
cacacaaagcttgcatttctcagcgctctggagatcgcg

SEQ ID NO: 12 - protein, 13 aa, Artificial sequence
  Synthetic sequence
KLAAALEHHHHHH

SEQ ID NO: 13 - protein, 14 aa, Artificial sequence
  Synthetic sequence
CKLAAALEHHHHHH

SEQ ID NO: 14 - protein, 5 aa, Artificial sequence
  Synthetic polyhistidine sequence
HHHHH

SEQ ID NO: 15 - protein, 7 aa, Artificial sequence
  Synthetic polyhistidine sequence
HHHHHHH

SEQ ID NO: 16 - protein, 8 aa, Artificial sequence
  Synthetic polyhistidine sequence
HHHHHHHH

SEQ ID NO: 17 - protein, 9 aa, Artificial sequence
  Synthetic polyhistidine sequence
HHHHHHHHH

SEQ ID NO: 18 - protein, 10 aa, Artificial sequence
  Synthetic polyhistidine sequence
HHHHHHHHHH

(19) United States
(12) Patent Application Publication
(10) Pub. No.: US 2012/0251446 A1
(43) Pub. Date: Oct. 4, 2012

(54) Peptide Conjugates Comprising Polyhistidine Sequence and Free Cysteine and Their Uses in Imaging

(75) Inventors: Richard Tavare, Los Angeles, CA (US); Gregory Mullen, London (GB); Philip Blower, London (GB); Rafael Torres Martin de Rosales, London (GB); Jennifer Williams, London (GB)

(73) Assignee: KING'S COLLEGE LONDON, London (GB)

(21) Appl. No.: 13/390,673

(22) PCT Filed: Aug. 18, 2010
(86) PCT No.: PCT/GB10/01564

§ 371 Date: Jun. 15, 2012

(30) Foreign Application Priority Data
Aug. 18, 2009 (GB) 0914446.0

(51) Int. Cl. A61K 51/08; G01N 21/17; C07K 19/00; C12P 21/00

(52) U.S. Cl. 424/1.69; 435/69.7; 435/7.1; 530/350; 530/391.5

(57) ABSTRACT

Bioconjugates for use in imaging are disclosed include a linker sequence fused to a polypeptide capable of binding a target in a biological system. The linker sequence is designed so that it is capable of being radiolabelled, e.g. with a complex comprising a radionuclide, via a free cysteine residue and the polyhistidine sequence in the label that are both capable of simultaneously binding to a complex comprising the radionuclide. These interactions can improve significantly the rate and efficiency of radiolabelling compared to either protein with the His-tag or the free cysteine alone. Optionally, the free cysteine residue provides a site which can be covalently bonded to a moiety such as a second label, in a site-specific manner.

### FIELD OF THE INVENTION

The present invention relates to bioconjugates and their uses in imaging, and more particularly to bioconjugates of polypeptides and linker sequences that are capable of being site-specifically labelled.

### BACKGROUND OF THE INVENTION

Radiolabelled bioconjugates have been used for a range of imaging studies in which a protein, or a domain or fragment thereof, binds to a target in a biological system enabling it to be imaged using a suitable radionuclide. However, existing bioconjugates and the reactions used to make them suffer from a number of disadvantages that make them unsuitable for use in many in vivo or therapeutic applications.

Firstly, the reactions used to conjugate the radionuclide to the protein often employ reactive amino acids on the surface of the protein, such as lysine residues, and activate them with a bifunctional chelator so that they can react with the radionuclide, typically provided as a complex. However, the fact that multiple amino acid residues are activated means that the reaction is not site-specific as the radionuclide complex reacts with the protein at in a varying number of the modified sites, leading to a wide range of different products. In addition, the activated sites, such as thiolated lysine residues or free cysteine residues, react with each other to form new disulphide bonds, distorting the structure of the protein and causing a loss of tertiary structure and activity. It is further known that making even a single change to the amino acid sequence of a polypeptide, for example to introduce a reactive amino acid for conjugation, can easily destroy the ability of the polypeptide to bind to a target. Accordingly, these reactions often lead to a mixture of protein products in which the proportion that remains functional is generally low. This in turn means that a purification step is needed to obtain a conjugate having sufficient purity (e.g., above 95%) to be used in in vivo applications. These factors make existing bioconjugates unsuitable for use in many therapeutic applications, where a well characterised conjugate present at high purity in a composition is desirable. The fact that the conjugates are often prepared for immediate use from a kit also means that reactions that do not have high yields and do not lead to clearly defined products are not suitable as purification steps cannot be readily carried out by a medical practitioner.

One important application of bioconjugates for imaging is in the investigation of apoptosis. Apoptosis is an energy-dependent, genetically controlled process by which cell death is activated through an internally regulated suicide program (1). It results in the exposure of specific components of the inner leaflet of the plasma membrane, such as phosphatidylserine (PS), on the surface of the cell. In contrast to necrotic cell death, which occurs following exposure to high concentrations of endogenous or exogenous toxins, heat treatment, freeze-thawing or other immediately disruptive insults, apoptosis tends to occur during less intense, chronic tissue insult. The ability to non-invasively investigate and image apoptosis or necrosis, or “cell death,” in diseases such as cancer, heart disease or immune rejection, is an important goal in monitoring response to treatment or assessment of tissue injury, and may also be of value in acquiring a deeper understanding of the pathophysiology of disease. In particular, the use of imaging techniques such as single photon emission computed tomography (SPECT) and positron emission tomography (PET) are assuming an important role in cell death imaging (2).

A common method of detecting externalized PS is the use of PS-binding proteins such as Annexin V or the C2A domain of synaptotagmin I. These are amphipathic molecules that bind to PS in a Ca²⁺-dependent manner (3, 4). Neither Annexin V nor C2A can discriminate between PS on the outer or inner leaflet of lysed cells and hence neither can distinguish between apoptosis and necrosis (5, 6). Nevertheless, using a variety of radiolabelling strategies, labelled Annexin V and C2A have been evaluated in several preclinical apoptosis or cell death imaging studies, and Annexin V has also progressed to several clinical trials (7).

As generally discussed above, when radiolabelling small proteins such as these, it is often important to exert maximum control over the number and site of modifications to the molecule. This is starkly illustrated by the example of In-111-DTPA-N-TIMP-2, a radiolabelled recombinant protein designed for imaging of matrix metalloprotein (MMP) expression. The attachment of the first DTPA occurred at a single location and was accompanied by full retention of MMP binding; whereas attachment of a second led to complete loss of function (8). In the case of Annexin V, Tait et al. elegantly showed that non-specific “amine-directed” labelling of Annexin V led to reduction or abolition of PS affinity (9) and poor in vivo performance (10, 11). Until recently nearly all molecular imaging studies with Annexin V and C2A to date had been done using amine-directed bifunctional agents. In most cases the extent and statistical distribution of modification have not been measured, and it has not been quantitatively demonstrated that the bioconjugates retain full bioactivity compared with unmodified protein. In cases such as these, the detrimental effects are compounded because the lowest affinity (most heavily modified) protein molecules carry a disproportionately high radiolabel signal. The biodistributions of Annexin V derivatives were improved to some extent by refining the non-site-specific labelling methods, leading eventually to ^{99m}Tc-HYNIC-Annexin V (12,13) (HYNIC refers to hydrazinonicotinamide). However, in these cases, no evidence is provided to show that Annexin V was labelled in a site-specific manner.

Like Annexin V, C2A (˜16 kDa) is vulnerable to inactivation by inappropriate modification. The Ca²⁺ binding sites within C2A-domain are surrounded by positively charged amino acids, among them several Lys residues, that were shown by mutagenesis to also be involved in phospholipid binding (14, 15). Since Lys residues are the usual site of modification for radiolabelling, great caution in bioconjugate synthesis and careful characterization of the products are required. C2A was first used as an MR cancer imaging agent in the form of a glutathione-S-transferase (GST) fusion protein which forms a non-covalent dimer of ˜85 kDa. The GST-C2A protein was non-specifically modified and covalently linked via Lys residues to either superparamagnetic iron oxide particles or the gadolinium complex of S-2-(4-isothiocyanatobenzyl)-DTPA (p-SCNBn-DTPA) (16, 17). This non-specific labelling method resulted in a decreased affinity for PS (18). For SPECT imaging, ^{99m}Tc labelled GST-C2A has been used in preclinical cardiac and cancer applications (19, 20). In these studies too, a non-site-specific method was employed: the fusion protein GST-C2A was treated with 2-iminothiolane to modify GST-C2A Lys amines to thiols then labeled with ^{99m}Tc-glucoheptonate.

WO 2003/044041 relates to alpha-fetoprotein conjugates and their uses for imaging. US Patent Application Publication 2004/0265392 relates to conjugates for immobilizing tumour necrosis factor on the surface nanoparticles.

Given these problems, producing current good manufacturing practice recombinant protein conjugates with consistent batch-to-batch quality using non-site-specific modification for clinical imaging is problematic. Removal of any inactive or low affinity protein prior to injection, if achievable at all, requires additional affinity chromatography purification steps (17). A cGMP cell death imaging agent developed for clinical application should be labelled site-specifically, reproducibly, efficiently and at room temperature. Preferably it should be using a simple kit-based method providing a well-characterized, homogeneous, fully functional and stable product. Tait et al. approached this objective by genetically engineering a derivative with an N-terminal amino acid AGGCGH tag, which can be labelled with ^{99m}Tc (9, 21). Others have recently genetically engineered a free Cys for the site-specific modification and radiolabelling of Annexin V (22).

Accordingly, it remains a problem in the art to provide labelled bioconjugates that overcome one or more of these disadvantages, and in particular to find bioconjugates that are suitable for use in in vivo imaging studies such as cell death imaging.

### SUMMARY OF THE INVENTION

Broadly, the present invention relates to bioconjugates that include a linker sequence fused to a polypeptide of interest, where the linker sequence is designed so that it is capable of being radiolabelled, e.g. with a complex comprising a radionuclide, and optionally covalently bonded to a moiety such as a second label, in a site-specific manner. This allows the production of a range of bioconjugates suitable for use in imaging studies, including in vivo cell death imaging and multi-modal imaging applications. Accordingly, the present invention enables a linker sequence that is capable of binding to a radionuclide to be built into a biological molecule and avoids the need to add a bifunctional chelator to activate the molecule as done in the prior art, with the consequential disadvantages mentioned above. The bioconjugates interact with the label(s) because the linker sequence incorporates both a polyhistidine tag (His-tag) and an additional free cysteine residue. The polyhistidine tag enables the bioconjugate to be purified using immobilized metal affinity chromatography (IMAC) purification and then to be site-specifically labelled with a radionuclide complex, e.g. with ^{99m}Tc or ¹⁸⁸Re using [(H₂O)₃M(CO)₃] (M=^{99m}Tc or ¹⁸⁸Re). The free cysteine residue can be used for site-specific covalent modification with prosthetic groups (labels) for optical or radiolabelling. In particular, the results disclosed herein show that the free cysteine residue and the polyhistidine tag are capable act synergistically to improve significantly the rate and/or efficiency of radiolabelling compared to either protein with the His-tag or the free cysteine alone. Accordingly, in the present invention both the free cysteine and the polyhistidine sequence are capable of simultaneously interacting with the radionuclide, thereby to improve the rate and/or efficiency of binding to conjugate to the radionuclide. The presence of a histidine tag has the further advantage that it is non-immunogenic and does not hinder the clinical development of histidine tag-containing recombinant proteins.

Accordingly, in a first aspect, the present invention provides a bioconjugate for use in imaging that comprises:
a polypeptide which is capable of interacting with a target of interest in a biological system; and
a linker sequence covalently bonded to the polypeptide, the linker sequence comprising (a) a free cysteine residue and (b) a polyhistidine sequence which is capable of site-specific labelling with a radionuclide for imaging the target of interest using the bioconjugate;
wherein both the free cysteine residue and the polyhistidine sequence are capable of simultaneously interacting with the radionuclide.

Generally, the polyhistidine sequence is capable of site-specific labelling with a complex comprising the radionuclide. In some aspects, the bioconjugate will have been reacted so that it is site-specifically labelled with the radionuclide and/or the second label. Accordingly, the present invention also provides the above bioconjugate in which the polyhistidine sequence is labelled with a radionuclide and/or the label is covalently linked to the linker sequence by a reaction with the free cysteine residue, for example via a reaction in which the free cysteine is covalently bonded to a label via a sulfhydryl-reactive group of the label reacting with the free thiol group of the cysteine residue. Examples of reactive groups include maleimide, haloacetyl (e.g. bromo- or iodo-), pyridyldisulfide and vinyl sulfone groups. Preferred examples of labels include fluorescent labels, MRI contrast agents, small molecule drugs and toxins.

In a further aspect, the present invention provides a kit for making a labelled bioconjugate for use in imaging employing a polypeptide which is capable of interacting with/binding to a target of interest in a biological system, the kit comprising:
(i) a linker sequence for covalently linking to the polypeptide, the linker sequence comprising (a) a free cysteine residue and (b) a polyhistidine sequence which is capable of site-specific labelling with a radionuclide for imaging the target of interest using the bioconjugate, wherein both the free cysteine residue and the polyhistidine sequence are capable of simultaneously interacting with the radionuclide, or a nucleic acid sequence encoding the linker sequence for ligation to a nucleic acid sequence encoding the polypeptide;
(ii) optionally, a complex comprising a radionuclide for labelling the polyhistidine sequence;
(iii) optionally, a label for site-specific reaction with the free cysteine residue of the linker; and
(iv) optionally, one or more additional reagents for carrying out the radiolabelling or labelling reactions.

In a further aspect, the present invention provides the use of a linker sequence for making a labelled bioconjugate for use in imaging, wherein the bioconjugate comprises a polypeptide which is capable of interacting with a target of interest in a biological system and the linker sequence is for covalent linkage to the polypeptide, the linker sequence comprising (a) a free cysteine residue and (b) a polyhistidine sequence which is capable of site-specific labelling with a radionuclide for imaging the target of interest using the bioconjugate, wherein both the free cysteine residue and the polyhistidine sequence are capable of simultaneously interacting with the radionuclide, or a nucleic acid sequence encoding the linker sequence for ligation to a nucleic acid sequence encoding the polypeptide.

In a further aspect, the present invention provides a method of making a bioconjugate for use in imaging, the method comprising:
(i) expressing a fusion protein of a polypeptide which is capable of interacting with/binding to a target of interest in a biological system and a linker sequence comprising (a) a free cysteine residue and (b) a polyhistidine sequence which is capable of site-specific labelling with a radionuclide for imaging the target of interest using the bioconjugate, wherein both the free cysteine residue and the polyhistidine sequence are capable of simultaneously interacting with the radionuclide;
(ii) contacting the fusion protein with the radionuclide (e.g. in the form of a complex) so that the radionuclide binds to the polyhistidine sequence;
(iii) optionally reacting the free cysteine residue and the label so that the label covalently bonds to cysteine residue.

In a further aspect, the present invention provides a bioconjugate as disclosed herein for use in a method of imaging.

In a further aspect, the present invention provides a method of imaging employing a bioconjugate of the present invention, the method comprising:
(i) introducing the bioconjugate into the biological system comprising the target of interest;
(ii) detecting the radionuclide to image the target of interest in the biological system; and
(iii) optionally detecting the label.

Embodiments of the present invention will now be described by way of example and not limitation with reference to the accompanying figures.

### BRIEF DESCRIPTION OF THE FIGURES

FIG. 1. Size exclusion, SDS/PAGE and ES-MS characterization of C2AcH. (A). After expression and purification by IMAC and heparin affinity chromatography, C2AcH was further purified by preparative size exclusion chromatography. In fast protein liquid chromatography (FPLC) size exclusion chromatogram shown, a discrete single peak with a retention time of 71 min as expected for a globular ˜16 kDa protein compared to molecular weight standards (data not shown). (B) The single peak obtained from the preparative size exclusion chromatography was analyzed by reduced (R) and non-reduced (NR) SDS/PAGE electrophoresis. In the SDS/PAGE gel shown, a single band under both the non reduced and reduced conditions of ˜16 kDa was observed as compared to molecular weight markers (M). This indicates that C2AcH exists as a single species and no dimer is present.

FIG. 2. Confocal microscopy of apoptotic mouse macrophages stained with C2AcH-F and/or anti-Caspase 3. C2AcH was site specifically modified at the Cys with fluorescein-maleimide. To show that C2AcH-F was functional and recognized apoptotic cells, C2AcH-F (panel A and B) was incubated with etoposide-treated macrophages in the presence (panel A, C and E) and absence (panel B, D and F) of 4 mM calcium. Cells were then permeabilized and stained for a specific intracellular marker of apoptosis, using rabbit anti-Caspase 3 followed by an Alexa Fluor 546 goat-anti-rabbit secondary antibody (panel C and D). The C2acH-F (green) images and anti-Caspase 3 (red) images were overlayed (panel E and F). C2Ac-F and anti-Caspase 3 did not bind to live (non apoptotic) cells.

FIG. 3. Radiolabeling, Size exclusion purification, SDS/PAGE and Phosphor image analysis of C2AcH[^{99m}Tc(CO)₃]. (A) C2AcH was radiolabelled with [(H₂O)₃^{99m}Tc(CO)₃]⁺ and purified on a PD-10 size exclusion column to remove any unincorporated radiolabel. In the graph shown, the eluted fractions (1 mL) were collected and the activity (MBq) per fraction was determined. (B) The labelled protein fractions 1 and 2 and unincorporated radiolabel fraction 5 and 6 from the PD-10 size exclusion column were then analyzed by SDS/PAGE electrophoresis under non-reducing conditions with molecular weight markers (M). In the SDS/PAGE gel shown, no activity was observed in lanes corresponding to fraction 5 and 6 presumably because the lower molecular weight radioactive species runs off the gel. (C) ^{99m}Tc radioactive markers were placed at the and 20 kDa molecular weight protein markers on the SDS/PAGE gel which and the image shown was acquired using a medium MultiSensitive Phosphor Screen for 30 s and analyzed using a phosphor imager (PerkinElmer Cyclone).

FIG. 4. Radiolabeling efficiencies of C2AcH and C2AcH-A. (A) The C2AcH (solid line) and C2AcH-A (dashed line) were incubated with [(H₂O)₃^{99m}Tc(CO)₃]⁺ at 10° C. for 120 minutes at a protein concentration of 100 μg (open symbol) and 10 μg (closed symbol). (B) The C2AcH (solid line) and C2AcH-A (dashed line) were incubated with [(H₂O)₃^{99m}Tc(CO)₃]⁺ at 37° C. for 120 minutes at a protein concentration of 100 μg (open symbol) and 10 μg (closed symbol)

FIG. 5. [(H₂O)₃Re(CO)₃] inhibits formation of dimeric C2AcH by binding to Cys. (A) C2AcH was incubated with and without [H₂O)₃Re(CO)₃]+ and shown is a non-reducing SDS/PAGE gel of samples after overnight incubation. M indicates molecular weight markers, and monomeric and dimeric C2AcH protein bands are indicated. (B) The monomer and dimer protein bands of C2AcH with and without [(H₂O)₃Re(CO)₃]⁺ incubation were quantified using the ImageJ image analysis software. The data represent the mean of three samples with standard error bars.

FIG. 6. Site specifically radiolabelled C2AcH binds to PS in a calcium dependent manner on RBC. Radiolabelled C2AcH (circles) or C2AcH-B (triangles) were incubated with preserved RBC in increasing calcium concentrations up to 10 mM. The cells were then washed and treated with 10 mM EDTA and the activity eluted was counted using a gamma counter. The data is shown as the mean of three replicates with standard deviation error bars.

FIG. 7. Scheme showing the labelling of a C2A domain of Synaptotagmin I with a radionuclide complex (in the example [M(CO)₃]⁺, where M=^{99m}Tc or Re) and/or a fluorescent label (in the example, fluorescein).

FIG. 8. Trypsin digest of C2AcH and C2AcH⁻[Re(CO)₂]⁺. UV chromatograms of (A) C2AcH and (B) C2AcH⁻[Re(CO)₃]⁺ when digested in solution with trypsin and analyzed using LC-MS. Filled arrows in the UV chromatograms show a peak that corresponds to the peptide LAAALEHHHH which has decreased in size upon incubation with [Re(CO)₂]⁺. Open arrows in the UV chromatograms highlight the presence of multiple new peaks that have appeared due to incubation with [Re(CO)₃]⁺. (C) The +MS of one of these new peaks in (B) corresponds to LAAALEHHHHHH-[Re(CO)₃]⁺ and has the characteristic rhenium isotope pattern.

FIG. 9. Extracted ion chromatograms for C2AcH⁻[Re(CO)₂]+ digested with trypsin and analyzed by LC-MS. (A) DV chromatogram of C2AcH⁻[Re(CO)₃]⁺ when digested with trypsin. The filled arrow in the UV chromatogram shows a peak that corresponds to the peptide LAAALEHHHH and the open arrow highlights the presence of multiple new peaks that have appeared due to incubation with [Re(CO)₂]⁺. (B) Extracted ion chromatogram of MW=840 corresponding to LAAALEHHHHHH⁻[Re(CO)₃]⁺. (C) Extracted ion chromatogram of MW=956 corresponding to (CK+LAAALEHHHHHH-[Re(CO)₃]⁺) or (CKLAAALEHHHHHH⁻[Re(CO)₃]⁺+H₂O).

FIG. 10. A scheme of [Re(CO)₃]⁺ binding to the polyhistidine portion of a linker of the present invention (CKLAAALEHHHHHH).

### DETAILED DESCRIPTION

### Polypeptides for Making Bioconjugates

The bioconjugates of the present invention may be formed using any suitable polypeptide or protein, or a fragment or domain thereof. Accordingly, while for convenience the methods herein are generally described by reference to “polypeptides”, this should be taken to include shorter sequences of amino acids (e.g., from 5 or 10 amino acids in length to 30, 40 or 50 amino acids in length), sometimes referred to in the art as peptides. The term should also be taken to include polypeptides having secondary, tertiary or quaternary structure, generally referred to as proteins, as well as multidomain proteins. In general, the conjugates of the present invention are intended to be formed such that the polypeptides used substantially retain tertiary structure, and hence retain substantially all of the properties of the unconjugated polypeptide. In some embodiments, and in the examples provided below, the polypeptides of the bioconjugates of the present invention are protein domains. “Protein domains” are fragments of a full length protein that have the ability to retain structure independent of the full length protein, typically forming a stable and folded three-dimensional structure. Many proteins consist of several structural protein domains and it is common for a particular domain to be found in a range of related proteins. Protein domains vary in length from between about 25 amino acids up to 500 amino acids in length, or from 50 amino acids to 250 amino acids, or from 75 amino acids to 150 amino acids.

Generally, the property required of the polypeptide portion of the bioconjugate is that it is capable of interacting with and/or specifically binding to a target component present in a biological system that the bioconjugate is used to label or image. In many cases, the polypeptide and the target component may be members of a specific binding pair, that is a pair of molecules which have particular specificity for each other and which in normal conditions bind to each other in preference to binding to other molecules. Examples of specific binding pairs are well known in the art and include receptors and ligands, enzymes and substrates, and antibodies and antigens.

In the present invention, where the polypeptide is an antibody, this term describes an immunoglobulin whether natural or partly or wholly synthetically produced. The term also covers any polypeptide or protein comprising an antibody binding domain. Antibody fragments which comprise an antigen binding domain are such as Fab, scFv, Fv, dAb, Fd; and diabodies. It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP 0 184 187 A, GB 2,188,638 A or EP 0 239 400 A.

Antibodies can be modified in a number of ways and the term “antibody molecule” should be construed as covering any specific binding member or substance having an antibody antigen-binding domain with the required specificity. Thus, this term covers antibody fragments and derivatives, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP 0 120 694 A and EP 0 125 023 A. It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242; 423-426, 1988; Huston et al, PNAS USA, 85: 5879-5883, 1988); (viii) bispecific single chain Fv dimers (PCT/US92/09965) and (ix) “diabodies”, multivalent or multispecific fragments constructed by gene fusion (WO 94/13804; Holliger et al, P.N.A.S. USA, 90: 6444-6448, 1993). Fv, scFv or diabody molecules may be stabilised by the incorporation of disulphide bridges linking the VH and VL domains (Reiter et al, Nature Biotech, 14: 1239-1245, 1996). Minibodies comprising a scFv joined to a CH3 domain may also be made (Hu et al, Cancer Res., 56: 3055-3061, 1996).

In one example of an application of the bioconjugates of the present invention, the polypeptide is capable of binding to phosphatidylserine (PS) so that the bioconjugate can be employed in apoptosis or cell death imaging studies. Examples of such polypeptides include Annexin V and the C2 domain of a polypeptide such as a synaptotagmin. Polypeptides that comprise one or more C2 domains are well known in the art. While some polypeptides have only one C2 domain, others have two or more C2 domains, and the domains are generally described by attaching a letter (in alphabetical order) to the end of the name (e.g., C2A, C2B, and so on). For a protein that contains only one C2 domain, the domain is simply referred to as C2 domain. While the examples below use the C2A domain of rat synaptotagmin I, other C2 domains that are capable of binding to PS could be employed instead, for example a C2A domain of a synaptotagmin of another species. Further examples of proteins that contain a C2 domain include but are not limited to synaptotagmin 1-13, protein kinase C family members of serine/threonine kinases, phospholipase A2, phospholipase 51, cofactors in the coagulation cascade including factors V and VIII, and members of the copine family. Human synaptotagmins include synaptotagmin 1-7, 12 and 13.

In some embodiments, the peptide is not alpha-fetoprotein or a variant thereof, for example as disclosed on WO2003/044041.

Other proteins that may be employed in the present invention include polypeptides capable of specifically binding to markers that are expressed by cancer cells, thereby enabling the cancer cells to be imaged using the bioconjugates. By way of example, the present invention can employ an anti-CD33 antibody, or fragment thereof, for imaging cancer cells expressing CD33 such as cells of myelomonocytic lineage and leukaemic cells, see Emberson et al., J. Immunol. Methods. 305(2):135-51, 2005. A further example is the use of a tissue inhibitor of metalloproteinases (TIMPs), such as TIMP-2, for imaging matrix metalloproteinase expression, as expression of metalloproteinases has been implicated in metastatic processes, see Giersing et al., Bioconjug Chem. 12(6): 964-71, 2001. A further example of a polypeptide that can be used to make conjugates according to the present invention is complement receptor 2 (CR2). Other possibilities include anti-CD169, anti-CD68 or anti-CD64 antibodies.

### Linker Construction

The linker sequences used in the conjugates of the present invention are designed so that they comprise a free cysteine for site-specific covalent modification and a polyhistidine tag for site-specific labelling, e.g. by interaction with a complex comprising a radionuclide. In this context, the skilled person can conveniently be able to determine with site-specific labelling has occurred by assessing the ability to label the bioconjugate without substantially interfering with its function. By way of example, the function of the bioconjugate will typically be the binding interaction of the polypeptide with the target component present in a biological system that the bioconjugate is used to image or label. In the present invention, “not substantially interfering with the function of the bioconjugate” preferably means that the radiolabelled bioconjugate will retain at least 60%, more preferably at least 75%, still more preferably at least 85%, and still more preferably at least 95%, of the binding affinity of the parent polypeptide used to form the bioconjugate for the target component of the biological system. The binding of the polypeptide or bioconjugate to the target component can be determined using techniques well known in the art for determining a binding affinity between a ligand and a receptor or a ligand and a target and include competitive ELISA, Biacore assay, cell binding assay, isothermal calorimetry or differential scanning calorimetry. This may be contrasted with prior art approaches in which modifying polypeptides at a varying number of amino acids for labelling leads to a plurality of different products, typically distorting the structure of the polypeptide and causing a loss of tertiary structure and function.

Experiments using the conjugates of the present invention found that the linkers may have two particular advantages. In some embodiments, the linkers enable the conjugate to be radiolabelled though the interaction of the polyhistidine tag with a complex comprising a radionuclide, while the free cysteine residue is capable of site-specific linkage to a label provides a second site for covalent linkage to a further label, thereby enabling multi-modal imaging studies to be carried out. Multi-modal imaging means that a single target component in a biological system, whether present in a sample or in vivo in a living organism, can be exposed to the bioconjugate in one experiment and two different types of imaging experiments carried out based on the detection of the radionuclide and the second label. This has the advantage that the polypeptide portion of the bioconjugate localises two labels at a site of interest in the biological system, enabling different information to be determined using the two labels.

The other unexpected advantage of the linkers of the present invention that is demonstrated in the examples is that the presence of the free cysteine increases the rate and/or efficiency of labelling the bioconjugate with the complex containing the radionuclide, i.e. where both the free cysteine residue and the polyhistidine sequence are capable of simultaneously interacting with the radionuclide, thereby enhancing its binding to the bioconjugate. In addition, this may involve increasing the yield of the radiolabelled bioconjugate and/or its specific activity and/or enabling the use of milder reaction conditions as compared to the use of the polyhistidine tag alone. This means that the linkers disclosed herein can also be used to improve bioconjugates in which the linker is radiolabelled and where a second label is not covalently attached to the free cysteine. The interaction of both moieties can be determined by those skilled in the art using experimental techniques such as tryptic digest analysis of radiolabelled bioconjugates and radiolabelling experiments. These techniques are used in the examples below to show that the free cysteine and the polyhistidine tag act synergistically to improve significantly the rate and efficiency of labelling with radionuclide containing complexes, such as [^{99m}Tc(CO)³]⁺ and [Re(CO)₃]⁺, compared to either protein with the His-tag alone, or the Cys alone, or neither.

Generally, the linker sequences will be between 6 and 25 amino acids in length, more preferably between 9 and 16 amino acids in length, and comprise a free cysteine residue, a polyhistidine sequence and, optionally, a sequence of amino acid residues (e.g. between 5 and 10 amino acid residues) between the free cysteine and polyhistidine sequence or at either end of the linker. In this context, “free cysteine” means that the cysteine residue does not participate in the formation of a disulphide bond with another cysteine present in the polypeptide sequence and is therefore capable of undergoing reactions to become covalently linked to the label and/or to interact with the complex comprising the radionuclide. The linker sequence can be provided at either or both of the N- or C-termini of the polypeptide, although it is often preferable to conjugate the linker to the C-terminus of the polypeptide as this reduces the tendency for the linker to affect the tertiary structure of the polypeptide part of the bioconjugate.

Accordingly, the polyhistidine sequence of the linker must be sufficiently long to be radiolabelled with a complex comprising the radionuclide. Generally, the use of polyhistidine sequences having between 5 and 10 histidine residues is preferred, and polyhistidine sequences having 5, 6 or 10 histidine residues are widely available as reagents. In some embodiments the linker sequence is represented by the general formula -Cys-Xₙ-His₅₋₁₀, where each X is any amino acid residue and n is between 5 and 10. When linked to a polypeptide, the resulting bioconjugates may be represented by the general formula polypeptide-Yₙ-Cys-Xₙ-His₅₋₁₀, where each Y and each X are independently any amino acid, m is between 0 and 10 and n is between 5 and 10. In the examples below, the linker comprises the sequence CKLAAALEHHHHHH.

### Methods of Producing the Bioconjugates

The unlabelled bioconjugates may be produced using methods well known to the skilled person. These include solid phase peptide synthesis and recombinant expression in a host cell using techniques well known in molecular biology. Solid phase peptide synthesis techniques are disclosed in Merrifield (J. Am. Chem. Soc., 85: 2149-2154, 1963) and a review of current techniques for the synthesis of peptides and proteins is provided in Kent (Annu. Rev. Biochem., 57: 957-959, 1988). In practice two strategies may be employed for solid-phase synthesis. Stepwise synthesis involves the addition of successive amino acids to the reactive C-terminal amino acid of a peptide coupled to a solid phase carrier. Fragment condensation involves the production of portions of a polypeptide by stepwise synthesis, that are then coupled together to provide the final polypeptide.

Alternatively, expression techniques may be used as shown in the examples to produce a fusion protein of the polypeptide and the linker sequence(s). Nucleic acid sequences encoding all or part of the fusion protein and any necessary regulatory elements can be readily prepared by the skilled person using techniques known in the art, for example, see Sambrook, Fritsch and Maniatis, Molecular Cloning, A Laboratory Manual, Cold Spring Harbour Laboratory Press, 1989, and Ausubel et al, Short Protocols in Molecular Biology, John Wiley and Sons, 1992. These techniques include the use of the polymerase chain reaction (PCR) to produce nucleic acid sequences encoding the fusion protein from template sequences. In order to express of the nucleic acid sequences encoding the fusion protein, the sequences can be incorporated in a vector having control sequences operably linked to these nucleic acid to control their expression. The vectors may include other sequences such as promoters or enhancers to drive the expression of the inserted nucleic acid, nucleic acid sequences so that the bioconjugate is expressed as a fusion and/or nucleic acid encoding secretion signals so that the polypeptide produced in the host cell is secreted from the cell. Suitable vectors can be chosen or constructed, containing appropriate regulatory sequences, including promoter sequences, terminator fragments, polyadenylation sequences, enhancer sequences, marker genes and other sequences as appropriate. Vectors may be plasmids or viral, e.g. ‘phage, or phagemid, as appropriate. The fusion protein may then be expressed transforming the vectors into host cells in which the vector is functional, culturing the host cells so that the fusion protein is produced and recovering it from the host cells or the surrounding medium. Prokaryotic and eukaryotic cells are used for this purpose in the art, including strains of E. coli, insect cells (e.g. transformed with baculovirus), yeast, and eukaryotic cells such as COS or CHO cells. Following production by expression, the fusion protein may be isolated and/or purified from the host cell and/or culture medium, as the case may be, and subsequently used as desired

### Radionuclides

The bioconjugates of the present invention are capable of being labelled with a radionuclide, for example a radionuclide provided as a complex. In general, the complexes react with the polyhistidine sequence of the linker so that some of the histidines act as ligands to the complex, replacing some of the ligands initially present. In the model system disclosed in the examples, a complex represented by the formula [(H₂O)₃M[(CO)₃]⁺, where M is a radionuclide preferably selected from ^{99m}Tc, ^{94m}Tc or ¹⁸⁸Re is used to label the bioconjugate. Examples of radionuclides that are chelatable by the compounds of the present invention include technetium, rhenium, copper, cobalt, gallium and indium isotopes such as Tc-99m, Re-186, Re-188, Co-57, Ga-67, In-111 (SPECT), Cu-64, Cu-60, Cu-61, Cu-62, Cu-67, Tc-94m, Ga-68, Co-55 (PET). The present invention may employ the radionuclides alone or in combinations. In general, technetium isotopes are employed for imaging purposes, rhenium isotopes for therapeutic purposes and copper isotopes for both imaging and therapy.

### Cysteine-Linked Labels

The presence of a free cysteine group in the linker enables the bioconjugates of the present invention to be covalently linked to a wide range functional moieties that include labels such as fluorescent labels, MRI labels, small molecule drugs or toxins.

In the case of fluorescent labels, there are established techniques for linking fluorescent labels to a reactive thiol group, e.g. as present in the linkers of the present invention, for example using fluorescent dyes having maleimide groups that react with free thiol groups. Examples of such fluorescent labels include DACM [N-(7-Dimethylamino-4-methylcoumarin-3-yl)maleimide], EDANS C2 maleimide, Fluorescein-5-maleimide, and maleimide derivatised HiLyte Fluor™ fluorescent labels.

The most commonly used MRI agents are intravenous contrast agents are based on chelates of gadolinium, for example formed with DOTA or DTPA. This may be achieved by reacting maleimide-DOTA or maleimide-DTPA with gadolinium and then conjugating it to the polypeptide-linker fusion. This sequence of steps has the advantage of avoiding the comparatively harsh conditions that are required to label the DOTA or DTPA with Gd, i.e. heating for 2 hours.

### Applications of the Bioconjugates

The applications of the bioconjugates of the present invention include a wide range of imaging and spectroscopic applications that can employ the radionuclide and/or the second label. As described herein, the bioconjugates are particularly useful for in vivo imaging applications such as cell death imaging, for example using bioconjugates for the detection of apoptosis. This might be useful in a number of different medical or research applications, for example in the fields of oncology, cardiovascular medicine (e.g. in imaging damaged myocardium post myocardial infarction) or graft rejection (e.g. in imaging cardiac allograft rejection).

The present invention is particularly relevant to nuclear medicine imaging techniques, such as Single Photon Emission Computed Tomography (SPECT), an imaging technique that detects gamma rays emitted from a radionuclide to produce a two dimensional image of the distribution of the radionuclide in a sample or subject, and Positron Emission Tomography (PET), an imaging technique that three-dimensional images by detecting pairs of gamma rays emitted indirectly by a positron-emitting radionuclide introduced into a sample or subject. By way of example SPECT studies can be carried out using ^{99m}Tc and PET studies using ^{94m}Tc. The skilled person, however, will be aware of other suitable SPECT and PET radionuclides that can be employed in the present invention.

In embodiments of the present invention in which a second label that has been covalently linked to the free cysteine residue are employed, the bioconjugates of the present invention may be used in methods of multi-modal imaging, that is where information or images are derived from the detection of the radionuclide and the second label at the site in the biological system where the bioconjugate becomes localised, e.g. by a binding interaction between the polypeptide and the target component of the biological system. Multi-modal studies may need to take place in two steps, but generally employ the same sample so that spatial information obtained using the two technique can be compared.

### EXAMPLES

### Experimental Procedures

### Molecular Biology.

The pET-29b vector containing amino acid residues 140-267 of Rat Synaptotagmin I, the C2A domain, (provided by Bazbek Davletov) was used as a template for the polymerase chain reaction (PCR) and subsequent sub-cloning to create two variants, both without the inherent S-Tag domain in the vector: C2Ac (C2A with a single cysteine residue on the C-terminus) and C2AcH (C2A with a single cysteine residue on the C-terminus, a linker (KLAAALE) and a hexahistine tag). The two constructs used the same forward primer: 5′-CAC ACA CAT ATG GAG AAA CTG GGA AAG CTC CAA with the reverse primer for C2Ac: 5′-CAC ACA AAG CTT TCA GCA TTT CTC AGC GCT CTG GAG ATC GCG and for C2AcH: 5′-CAC ACA AAG CTT GCA TTT CTC AGC GCT CTG GAG ATC GCG. The resulting PCR fragments were sub-cloned back into the pET-29b vector using the restriction enzymes NdeI and HindIII and sequenced (King's College London, Molecular Biology Unit, UK). After sequence confirmation, DNA constructs for C2Ac and C2AcH were transformed into the BL21 (DE3) strain of Escherichia coli. For both constructs, cultures in 10 mL of L-broth containing 50 μg/mL kanamycin (Sigma, Gillingham, UK) were grown overnight at 37° C. with agitation, and then added to 1000 mL L-Broth and grown at 37° C. with agitation until the OD₆₀₀ reached 0.8. Expression was then induced by adding 1 mM isopropyl β-D-1-thiogalactopyranoside (IPTG) (Merck Chemicals, Nottingham, UK) followed by further incubation at 37° C. with agitation for 4 hours. Cells were then pelleted by centrifugation at 4,000 RCF for 10 min. at 4° C. and then frozen at −80° C. Pellets were resuspended in 50 mL Resuspension Buffer (RB, 20 mM Tris-HCl pH 7.5, 150 mM NaCl, 1 mM EDTA, 0.1% TritonX-100 and Complete Protease Inhibitors (Roche Diagnostic, Burgess Hill, UK)). Suspensions were then sonicated four times for 15 s with 1 min. intervals on ice and allowed to recover for 30 min. on ice. The suspension was then centrifuged at 4° C. for 20 min. at 17,200 RCF. For C2AcH purification, the supernatant was added to a 1 mL Nickel column (GE Healthcare, Amersham, UK) at 1 mL/min using an AKTA FPLC (GE Healthcare, Amersham, UK) which had previously been equilibrated with nickel binding buffer (NBB, 20 mM Tris-HCl pH 7.5, 150 mM NaCl). The column was then flushed with 10 column volumes (CV) of NBB and washed with 20 CV of nickel wash buffer (NWB, 20 mM Tris-HCl pH 7.5, 150 mM NaCl, 50 mM imidazole). The protein was then eluted with 20 CV of nickel elution buffer (NEB, 20 mM Tris-HCl pH 7.5, 150 mM NaCl, 500 mM imidazole). Elution fractions containing protein were dialyzed overnight against 3 L NBB using a 7 kDa molecular weight cut-off dialysis tubing at 4° C. with gentle stirring. 4 mM CaCl₂ was then added to dialyzed protein before addition to a 5 mL Heparin Column (GE Healthcare, Amersham, UK) which had previously been equilibrated with Heparin Binding Buffer (HBB, 20 mM Tris-HCl, 150 mM NaCl, 2 mM CaCl₂, pH 7.5) at 1 mL/min. The column was washed with 10 CV of HBB and the protein was then eluted with 10 CV of Heparin Elution Buffer (HEB, 20 mM Tris-HCl, 200 mM NaCl, 10 mM EDTA, pH 7.5). To elute any dimer that may have formed, the column was eluted with 10 CV of Heparin Clean Buffer (HCB, 20 mM Tris-HCl, 1.5 M NaCl, 100 mM EDTA, pH 7.5). Any dimer present in this eluate was reduced with 10 mM dithiothreitol (DTT) for 4 h at RT and then loaded on a Sephacryl 100 size exclusion column (60 cm height, GE Healthcare, Amersham, UK) equilibrated with PBS such that the load volume did not exceed 5% of a column volume. C2Ac was purified using the heparin procedure and the elution pool was loaded on a Sephacryl 100 size-exclusion column as described above. The gel permeation elution peak was collected and the concentration was determined by absorbance 280 nm with an extinction coefficient of 12210 M⁻¹ cm⁻¹. Purified proteins were analysed by SDS-PAGE, analytical HPLC-SEC and LC-MS (University of Kent, UK). C2AcH was purified at 30 mg per liter culture and C2Ac was purified at 60 mg per liter culture.

### Fluorescein-5-Maleimide, N-(Benzyloxycarbonyloxy) Succinimide and Iodoacetimide Conjugates of C2Ac and C2AcH.

C2Ac and C2AcH were conjugated to fluorescein-5-maleimide (Sigma-Aldrich, Poole, UK). 1 mL of C2AcH or C2Ac (2 mg/mL in PBS) were mixed with a 5 molar excess of fluorescein-5-maleimide dissolved in 50 μL of dimethylsulfoxide (DMSO) and incubating at room temperature for 4 h. Unreacted fluorescein-5-maleimide was removed using a PD-10 column which has been pre-equilibrated with PBS. PD-10 fractions were monitored by UV absorbance at 280 nm and analysed by SDS-PAGE and LC-MS. The fluorescein conjugates of C2Ac and C2AcH are referred to as C2Ac-F and C2AcH-F, respectively.

To generate a non Cys like mutant the thiol was modified with iodoacetimide. C2AcH was incubated at 2 mg/mL with a 1.5 molar excess of iodoacetimide (Sigma-Aldrich, Poole, UK) overnight at 4° C. Unreacted iodoacetimide was removed using a PD-10 column and protein fractions were analysed by SDS-PAGE and LC-MS.). The iodoacetimide conjugate of C2AcH is referred to as C2AcH-A. The free thiol content of C2AcH and C2AcH-I was determined by Ellman's Reagent by as described by manufacture (Pierce Biotechnology, Rockford, US) (29). To generate a non-specifically modified C2A protein for radiolabelling, C2AcH was derivatised with N-(benzyloxycarbonyloxy)succinimide (Sigma-Aldrich, Poole, UK) as described above except a 20 to 1 molar ratio of N-(benzyloxycarbonyloxy)succinimide to C2AcH was used with incubation for only 1 h, giving rise to C2AcH-B, and samples were analysed by SDS-PAGE.

### Flow Cytometry and Immunofluorescence.

J774.2 murine monocytes macrophage cell line (ECACC, Porton Down, UK) were grown in DMEM (Invitrogen, UK) supplemented with Penicillin (100 Units/mL), Streptomycin (100 μg/mL), L-glutamate (5 mM), fetal bovine serum (10%, Sigma), sodium pyruvate (1 mM) and HEPES (10 mM). To test the binding capability of C2Ac and C2AcH-F to necrotic and apoptotic cells, the fluorescein conjugates were incubated with control or etoposide (MBL International, Woburn US) treated (15 μM, overnight at 1×10⁶ cells/mL) murine macrophages and compared to Annexin V-FITC (Invitrogen, Vybrant Apoptosis Kit) using a FACScalibur (Becton, Dickinson, Oxford UK) flow cytometry. Acquisition was performed according to commercial kit instructions.

For confocal microscopy, macrophages were plated at 5×10⁵ cells per well in a 6-well cell culture plate on sterile glass coverslips. On day 2, cells were treated overnight with 15 μM etoposide or PBS control. Cells were washed 3 times in PBS and then 3 times in either cell binding buffer (CBB, 10 mM HEPES, 140 mM NaCl, 2 mM CaCl₂, pH 7.4) or cell non-binding buffer (CNBB, 10 mM HEPES, 140 mM NaCl, 10 mM EDTA, pH 7.4). Coverslips were then incubated for 15 min in 200 μL of CBB or CNBB containing 2 μL propidium iodide (PI) and 3 μL of 0.6 μM C2AcH-F, washed 3 times with CBB or CNBB then 3 times in PBS, fixed with 4% PFA for 10 min. at room temperature (RT) and washed a further 3 times in PBS. For immunostaining, cells were permeabilized in 0.1% Triton X-100 in 10% FBS for 10 min. at RT. Anti-cleaved-caspase-3 antibody (Cell Signalling Technology) was diluted 1:200 in 2% FBS and incubated with cells for 1 h at RT. Coverslips were washed 3 times in PBS and then incubated with Alexa Fluor 546 goat-anti-rabbit (Invitrogen) diluted 1:200 in 2% FBS for 1 h at RT. Coverslips were again washed 3 times in PBS, then 3 times in water before being mounted on a slide with fluorescent mounting medium (Dako). Slides were analysed by confocal microscopy.

### Rhenium Labelling of C2AcH and C2AcH-A.

Rhenium tricarbonyl (fac-[(H₂O)₃Re(CO)₃]Br) was prepared and characterized as previously reported (30). Briefly, [Re(CO)₅]Br was refluxed in distilled H₂O for 24 h. The crude mixture was filtered and the solution concentrated under vacuum to give fac-[(H₂O)₃Re(CO)₃]Br as a light green powder in nearly quantitative yield. The final product was characterized by IR and ES-MS. C2AcH or C2AcH-A was labelled with rhenium by incubating 100 μg of C2AcH or C2AcH-A in 100 μL of PBS with a ten fold molar excess of [(H₂O)₃^{187/185}Re(CO)₃]⁺. This mixture was left to incubate at 37° C. for 30 min. before being passed through a PD-10 column (Sephadex G-25, GE Healthcare). The protein was analysed by SDS-PAGE and ES-MS.

To investigate whether rhenium tricarbonyl bound to the Cys, C2AcH was incubated with or without [(H₂O)₃Re(CO)₃]Br as described above. After reacting, a 100 μL of PBS pH 8.0 was added to each of the C2AcH solutions and left overnight at 37° C. The formation of a dimer was monitored by running equal amounts (as determined by A₂₈₀) of C2AcH with and without [(H₂O)(CO)₃]Br on a non-reduced SDS/PAGE gel. Monomeric (˜16 kDa) and dimeric (˜32 kDa) of C2AcH bands were quantified using ImageJ analysis software (NIH, Bethesda, US).

In order to map the rhenium tricarbonyl specific binding to the C-terminal CKLAAALEHHHHHH moiety a tryptic digest was performed on rhenium tricarbonyl labelled and unlabelled C2AcH and C2AcH-A using a method as previously described (8) and analyzed by LC-MS using a TK.

In order to map the rhenium tricarbonyl specific binding to the C-terminal CK-LAAALEHHHHHH moiety a tryptic digest was performed on rhenium tricarbonyl labeled and unlabelled C2AcH and C2AcH-A. 100 μg of proteins at 1 mg/mL in PBS were dissolved in 25 μL of 8 M urea in 100 mM ammonium carbonate. Urea was diluted by the addition of 75 μL dH₂O to make a final volume of 200 μL and a final concentration of 2 M urea. Proteins were then incubated with 10 μL of a 0.5 mg/mL solution of modified sequence grade trypsin (Promega, Southampton, UK) in solution for 3 h at RT. Digests were analyzed by LC-MS immediately after digestion.

LC/MS analysis of tryptic digests used a Vydac 218TP C-18 column with a 2.1 mm internal diameter at a flow rate of 200 μL/min. Buffer A was dH₂O and 0.05% trifluoroacetic acid (TFA) and Buffer B was dH₂O with 70% acetonitrile and 0.045% TFA.

^{99m}Tc Labelling of C2Ac, C2AcH, C2Ac-F, C2AcH-F, C2AcH-A and C2AcH-B.

^{99m}Tc pertechnetate eluted with saline from a Drytec generator (GE Healthcare, Amersham, UK) was converted to [(H₂O)₃^{99m}Tc(CO)₃]⁺ using the Isolink kit® (Mallinckrodt-Tyco, Petten, The Netherlands) and quality control was carried out according to manufacturer's instructions using instant thin-layer chromatography (ITLC) and analysis with a gamma-ray TLC scanner (Lablogic, UK) and high performance liquid chromatography (HPLC series 1200, Agilent, UK) equipped with an inline gamma detector (Lablogic, UK). C2A proteins were labeled with ^{99m}Tc by incubating 100 μg of C2Ac, C2Ac-F, C2AcH, C2AcH-F, C2AcH-A or C2AcH-B in 100 μL of PBS with up to 700 MBq in 100 μL of [(H₂O)₃^{99m}Tc(CO)₃]⁺. Unless otherwise stated the specific activities for all species were performed at ˜0.4 MBq/μg. However, to reproduce a clinically relevant specific activities C2AcH was also labelled at 7 MBq/μg. This mixture was left to incubate at either 10, 20 or 37° C. for up to 120 min. before being passed through a PD-10 column (Sephadex G-25, GE Healthcare). Labelling efficiency was calculated by comparing the amount of radioactivity associated with the eluted protein fraction versus unincorporated eluted (low molecular weight) radioactivity using a gamma counter (LKB Wallac, 1282 COMPUGAMMA) or dose calibrator (CRC-25R, Capintec, US). It should be noted that up to ˜5% of activity remains bound to the PD-10 column and therefore radiolabelling efficiency was also confirmed by ITLC. The same ITLC conditions were used as described above and the radiolabelling efficiency was calculated as a ratio of the radiolabelled protein peak (R_{f}=0) vs unincorporated [(H₂O)₃^{99m}Tc(CO)₃]⁺ ((R_{f}=0.9). Binding of radioactivity to protein was also confirmed by SDS-PAGE of the protein fraction followed by electronic autoradiography of the gel (Cyclone phosphorimager, Perkin Elmer, UK).

Binding of ^{99m}Tc Labelled C2AcH and C2AcH-B to PS on Red Blood Cells.

The binding of radiolabelled C2AcH to PS on red blood cells (RBC) was performed according to a literature method (31). A commercial preparation of preserved human RBC was obtained from Beckman-Coulter (High Wycombe, UK). Calcium titrations of RBC were performed in a buffer of 50 mM HEPES-sodium, pH 7.4, 100 mM NaCl, 3 mM NaN₃, with 1 mg/mL BSA as carrier protein. Reactions were prepared with 1 nM of ^{99m}Tc-labelled C2AcH (at a specific activity of 3.5 MBq/μg) and calcium; RBC were then added and the reaction (1 mL) was incubated for 8 min. at room temperature. The cells were then centrifuged (3 min. at 7800 g), the supernatant was removed, and the cells were resuspended in 1 mL assay buffer containing the same concentration of calcium used during the incubation step. The cells were centrifuged again, the supernatant was removed, and the pellet was resuspended in 0.7 mL assay buffer plus 10 mM ethylenediaminetetraacetic acid (EDTA) to release calcium dependent bound ^{99m}Tc labelled C2AcH. After centrifugation to remove the RBC, the released ^{99m}Tc labelled C2AcH in the supernatant was measured using a gamma counter. The EC₅₀ was calculated as described in the literature using the equation Y=[Ca]^{N}/([Ca]^{N}+EC₅₀^{N}) (31) where Y=B/Bₘₐₓ, B is the observed amount of radiolabelled protein bound at a given calcium concentration, and Bₘₐₓ is the amount of radiolabelled protein bound at saturating calcium concentrations. Curve fitting was performed using a non-linear curve fit by a routine based on the Levenberg-Marquardt algorithm using Kaleidagraph (Synergy Software, Reading, US).

### Serum Stability.

C2AcH and C2AcH-F were labelled with 400 MBq [(H₂O)₃^{99m}Tc(CO)₃]⁺ in 100 μL for 30 min. or 1 h at 37° C. After purification on a PD-10 column, 100 μL of labelled C2AcH or C2AcH-F was added to 400 μL human serum (Sigma, Poole, UK). As a control, 100 μL of labelled C2AcH or C2AcH-F was also added to PBS. The samples were then incubated at 37° C. At 0, 3, 6, and 18 h samples were taken and analysed by ITLC using a mobile phase of methanol and 1% concentrated HCl. As a control a separate ITLC of [(H₂O)₃^{99m}Tc(CO)₃]⁺ and [TcO₄]⁻ was performed in the same mobile phase. ITLC were then monitored using a radio TLC scanner (LabLogic, UK). Serum stability was calculated as the area under the protein peak (Rf=0) versus the area under the curve of the remainder of the chromatogram ([(H₂O)₃^{99m}Tc(CO)₃]⁺ or [TcO₄]⁻ Rf=1.0).

### Biodistribution.

Adult male BL57c/6 mice were injected i.v. in the tail vein with about 2 MBq of [^{99m}Tc(CO)₃]-C2AcH in 100 μL (n=3). After 2 h, mice were culled and dissected for the following organs: heart, lungs, blood (20 μL), stomach, large intestines, small intestines, kidneys, spleen, liver, and tail. Each sample was weighed and radioactivity was counted using a gamma counter (LKB compugamma) with standards prepared from a sample of injected material. The percent of the injected dose per gram (% ID/g) of tissue was calculated for each tissue type.

SPECT Imaging.

Adult male BL57c/6 mice were injected i.v. in the tail vein with about 20 MBq of [^{99m}Tc(CO)₃]-C2AcH in 200 μL (n=3). Animals were anesthetized using isofluorane and imaged using a nanoSPECT/CT animal scanner (Bioscan Inc.). Static images were acquired at 30, 45, 60, 75, and 90 min. post-injection. Using the BatchTool Generator, the static images can be make into dynamic images by insuring that the colours in each image correspond to the same physical voxel values. This tool automates the process of assembling the SPECT data sets in which identical reconstruction parameters are desired. SPECT images were obtained in 20 projections over 15 min. using a 4-head scanner with 1 mm pinhole collimators in helical scanning mode. CT images were obtained with a 45 kVP X-ray source, 1000 ms exposure time in 180 projections over 10 min. Images were reconstructed using proprietary Bioscan InVivoScope (IVS) software.

### Results

### Cloning, Preparation and Characterization of C2Ac, C2AcH, C2AcH-F, C2AcH-A and C2AcH-B

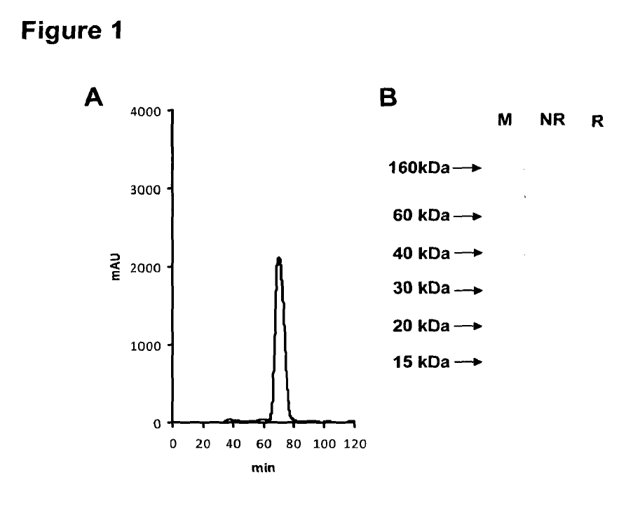

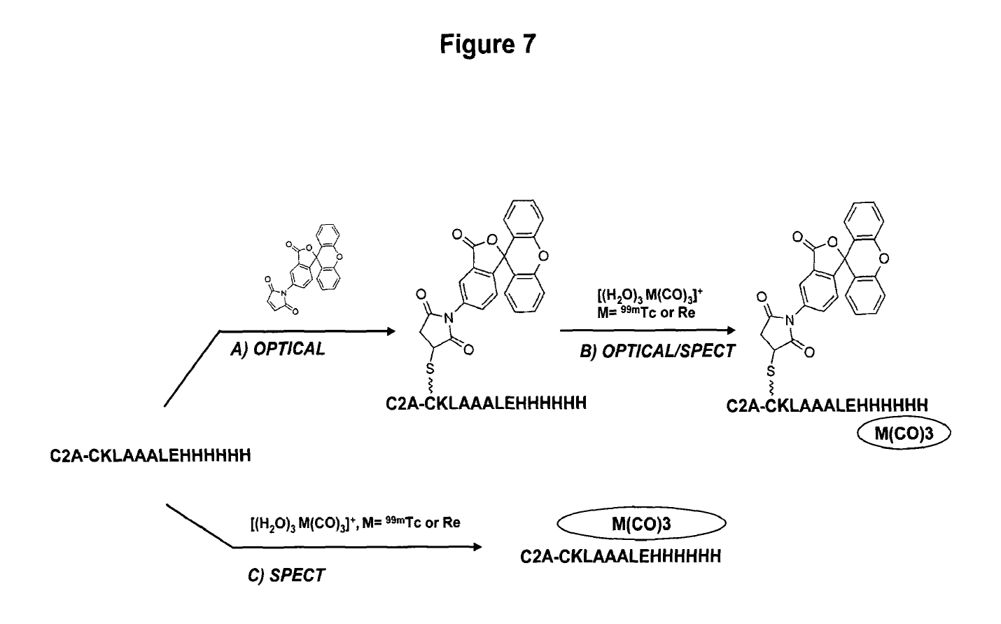

C2A was cloned into the pET 29d bacterial expression vector with the addition of a C-terminal site specific Cys with and without a His-tag, forming two constructs C2AcH (with His-tag) and C2Ac (without His-tag), respectively (FIG. 7). After induced expression in E. coli, C2Ac and C2AcH were isolated from E. coli in the soluble fraction. C2AcH was purified first via IMAC and further purified by heparin affinity chromatography in the presence of Ca²⁺, while C2Ac was purified directly using heparin affinity chromatography. C2Ac and C2AcH were buffer-exchanged and purified by size exclusion chromatography (FIG. 1a). C2AcH eluted as a single discrete peak with a retention time expected for a protein of ˜16 kDa, with no evidence of significant aggregation or dimerization. C2AcH was analyzed by reduced and non-reduced SDS-PAGE, giving rise to a single monomeric band at the expected molecular weight (FIG. 1b). The final protein products were analyzed by electrospray mass spectrometry (ES-MS) and were of the expected molecular weights (FIG. 1c and Table 1). C2AcH and C2Ac were modified with either fluorescein maleimide or N-(benzyloxycarbonyloxy)succinimide and iodoacetimide. C2AcH-F and C2AcH-A conjugates were analyzed by ES-MS and were of the expected molecular weights for the addition of one fluorescein as well as second minor peak in the liquid chromatography which by ES-MS is due to the addition of a second fluorescein molecule. However for C2AcH-A the addition of only one acetimide group, with no unconjugated C2AcH, was observed in the LC-MS (Table 1). The single modification at the Cys residue was confirmed by Ellmans' Reagent which showed no detectable free thiol in C2AcH-A while the expected amount of free thiol was observed in C2AcH.

On forming C2AcH-B, the benzyloxycarbonyloxy conjugate of C2AcH, a minor fraction of the protein precipitated. The soluble fraction did not give rise to any peaks in ES-MS and was therefore analyzed by non-reducing and reducing SDS/PAGE, giving rise to a single band at a lower molecular weight than C2AcH, indicating that the C2AcH-B had an altered conformation (data not shown).

TABLE 1
Summary of ES-MS data of C2Ac and C2AcH protein
conjugates and peptides post tryptic digest

| Protein (M)/ | Expected | Found |
|---|---|---|
| Peptide (P) | (Da) | (Da) |
| C2Ac | 14997 | 14998 [M + H]⁺ |
| C2AcH | 16517 | 16518 [M + H]⁺ |
| C2AcH-A | 16574 | 16575 [M + H]⁺ |
| C2Ac-F | 15542 | 15542 [M + H]⁺, 15524 |
| | | [M − H₂O]⁺; |
| | | 15869 [M + 2xF + H₂O]⁺ |
| C2AcH-F | 16944 | 16944 [M + H]⁺, 16962 |
| | | [M + H₂O]⁺ |
| | | 17389 [M + 2xF]⁺; 17407 |
| | | [M + 2xF + H₂O]⁺ |
| C2AcH-^{187/185}Re(CO)₃ | 16787 | 16787 [M]⁺ |
| LAAALEHHHHHH | 705 | 705 [P + 2H]²⁺ |
| LAAALEHHHHHH- | 840 | 840 [P + Re(CO)₃ + H]²⁺ |
| ^{187/185}Re(CO)₃ | | |
| CKLAAALEHHHHHH | 820 | Not Observed |
| CKLAAALEHHHHHH- | 956 | 956 [P + Re(CO)₃ + H]²⁺ |
| ^{187/185}Re(CO)₃ | | |

### C2AcH-F Binds to Apoptotic Cells in a Calcium Dependent Manner

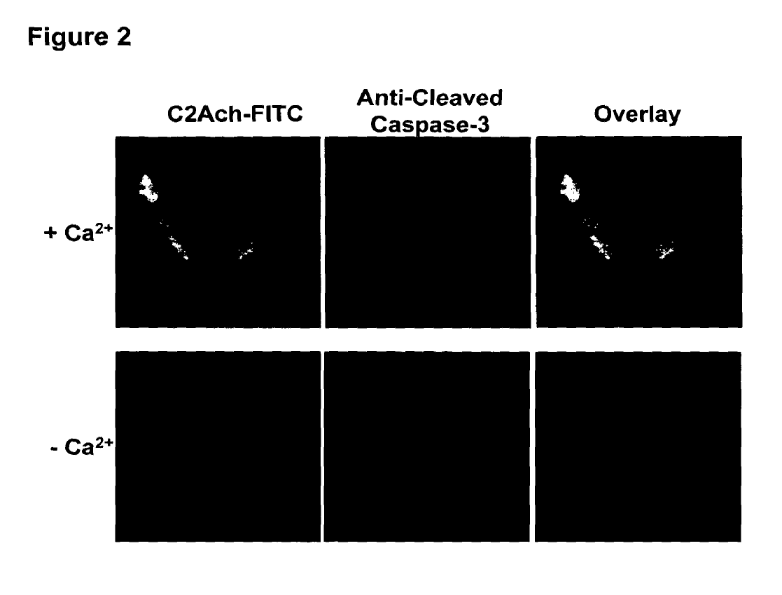

The ability of C2AcH-F to bind apoptotic cells was monitored by the incubation of C2AcH-F with live murine macrophages in culture that had been treated with etoposide (FIG. 2). After fixation, permeabilization and staining with anti-caspase 3, confocal microscopy confirmed that in the presence of calcium C2AcH-F binds to the extracellular membrane of caspase 3 positive cells, while in the absence of calcium there is no binding of C2AcH-F to the cell membrane. Therefore, after the site specific labelling of C2AcH with fluorescein via the cysteine thiol, it remained functional in a calcium dependent manner. Cells were also analyzed by flow cytometry by incubating live macrophages with C2AcH-F or Annexin V-FITC and similar results were observed.

Labelling of C2Ac, C2AcH, C2Ac-F, C2AcH-F and C2AcH-A with [^{99m}Tc(CO)₃]⁺ or [Re(CO)₃]⁺

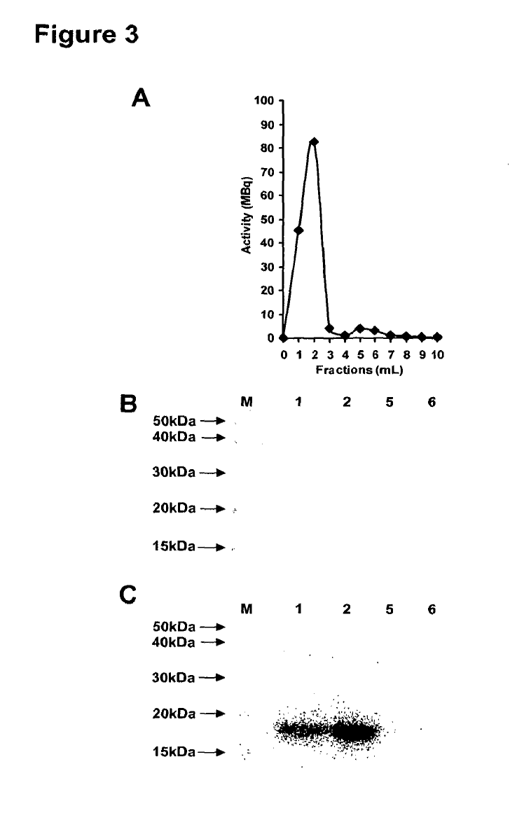

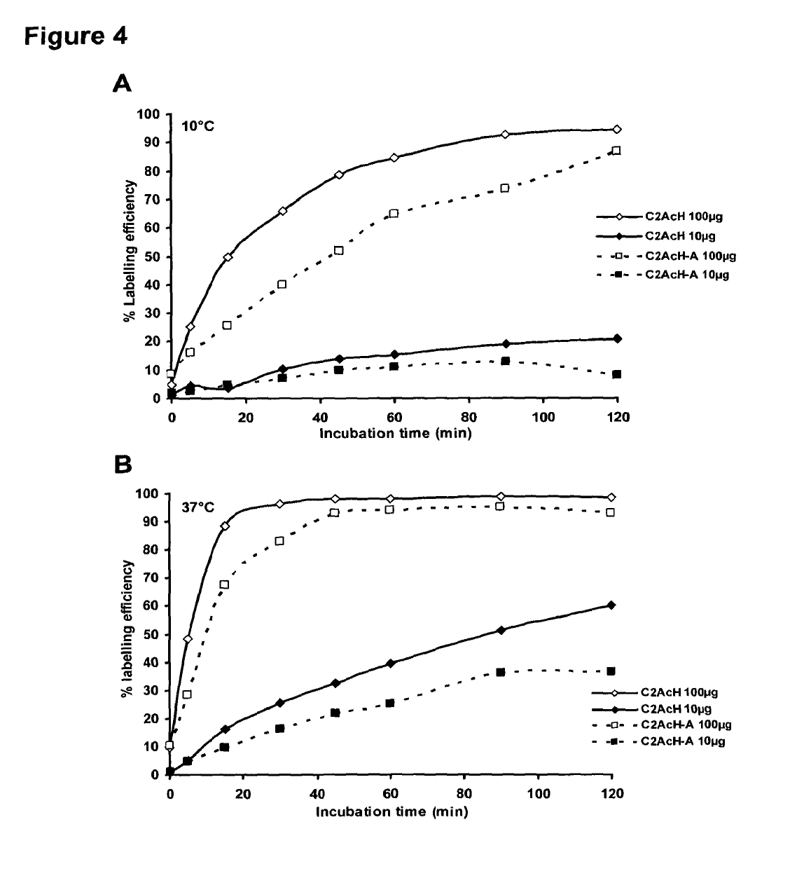

To compare the labelling efficiency of the proteins with [M(CO)₃]⁺ (where M. ^{99m}Tc or Re), C2Ac, C2AcH C2Ac-F, C2AcH-F and C2AcH-A were labelled by incubating either [(H₂O)₃⁹⁹° Tc(CO)₃]⁺ or ten fold excess of [(H₂O)₃Re(CO)₃]⁺ with the proteins (100 μg) at 10° C., 20° C. or 37° C. for up to 120 min. (Scheme 1). Radiolabelled proteins were purified on a PD-10 column and analysed by SDS/PAGE and autoradiography (FIG. 3). A radiochemical yield, as analyzed by ITLC, >95% was achieved with C2AcH at protein concentration of 1 μg/μL (which possesses both a free Cys and His-tag) at 37° C. within 30 min, (FIG. 4). The radiochemical purity was 100%. Longer incubation times did not improve the radiochemical yield. However, when a 10-fold lower protein concentration of C2AcH (0.1 μg/μg) was used only a radiolabelling yield of 25% was achieved at 37° C. which increased overtime to reach 98% after 24 hrs. When C2AcH was radiolabelled at the lower temperature of 10° C. for 30 min. a radiochemical yield of 65% and 10% was achieved at protein concentrations of 1 and 0.1 μg/μL respectively. In order to assess the contribution of the free Cys to the excellent radiolabelling efficiency of C2AcH, the Cys thiol was “blocked” using iodoacetimide. At a protein concentration of 1 μg/μg only a radiochemical yield of ˜83% at 37° C. and ˜40% at 10° C. at 30 min. was achieved with C2cH-A. While at a protein concentration of 0.1 μg/μg a radiochemical yield of 16% was achieved at 37° C. which increased overtime to reach 88% after 24 hrs (data not shown). When C2AcH-A was radiolabelled at the lower temperature of 10° C. for 30 min. a radiochemical yield of 40% and 7% was achieved at protein concentrations of 1 and 0.1 μg/μL respectively. When C2Ac (no his-tag) was radiolabelled a maximum radiochemical yield of <15%, which did not improve upon further incubation or increase in temperature (data not shown). When C2AcH and C2AcH-A were radiolabelled at higher and more clinically relevant specific activities (i.e. 7 MBq/μg) a radiolabelling efficiency of 94% and 88% was achieved at 37° C. at 30 min., respectively

Labelling of C2AcH and C2AcH-A with [Re(CO)]⁺ was confirmed by ES-MS and were of the correct molecular weight assuming that [Re(CO)₃]⁺ replaces a proton (Table 1). Despite the presence of a ten-fold excess of [(H₂O)₃Re(CO)₃]⁺ over protein, no evidence of proteins with more than one [Re(CO)₃]⁺ attached to a protein was seen in the ES-MS data, suggesting that the modification is site-specific. Furthermore, when a tryptic digest was performed on rhenium tricarbonyl labelled C2AcH and C2AcH-A only peptides containing the amino acid sequence CKLAAALEHHHHHH or KLAAALEHHHHHH contained the addition of one [Re(CO)₃]⁺ (Table 1).

Labelling of C2AcH and C2AcH-A with [Re(CO)]⁺ was confirmed by LC-MS and were of the correct molecular weight assuming that [Re(CO)₃]⁺ replaces a proton (Table 2). When a tryptic digest was performed on rhenium tricarbonyl labeled C2AcH and C2AcH-A, only peptides containing the amino acid sequence CKLAAALEHHHH or LAAALEHHHHHH contained the addition of one [Re(CO)₃]⁺ (Table 2). Interestingly, due to the presence of a lysine (K) between the cysteine and the His-tag, the His-tag and the cysteine can be separated by tryptic digest. When the free thiol is ‘blocked’ by acetimide, the C_{acetamide}K is not present in the extracted ion chromatograms. However, the addition of 18 Da to CKLAAALEHHHH-[Re(CO)₃]⁺ could be due to water binding to the molecule or to the strong binding of CK to the [Re(CO)₃]⁺ after tryptic digest. This weight of 965 Da leads to the possibility that CK could remain bound to [Re(CO)₃]⁺ after tryptic digest and provides further support that the thiol is involve in [Re(CO)₃]+ or [^{99m}Tc(CO)₃]⁺ binding. The presence of CKLAAALEHHHH-[Re(CO)₃]⁺ could be due to a conformational change upon [Re(CO)₃]⁺ binding that does not allow trypsin to digest at that lysine cleavage site. The reaction of C2Ac with [Re(CO)₃]⁺ under these conditions did not give rise to a detectable rhenium conjugate and only unmodified C2Ac was detected by LC-MS.

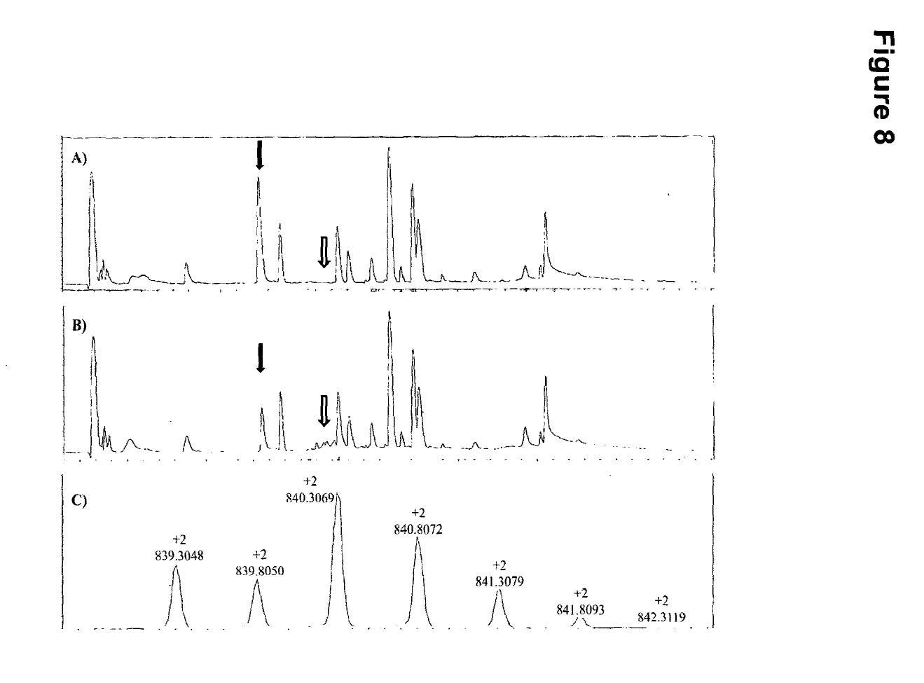

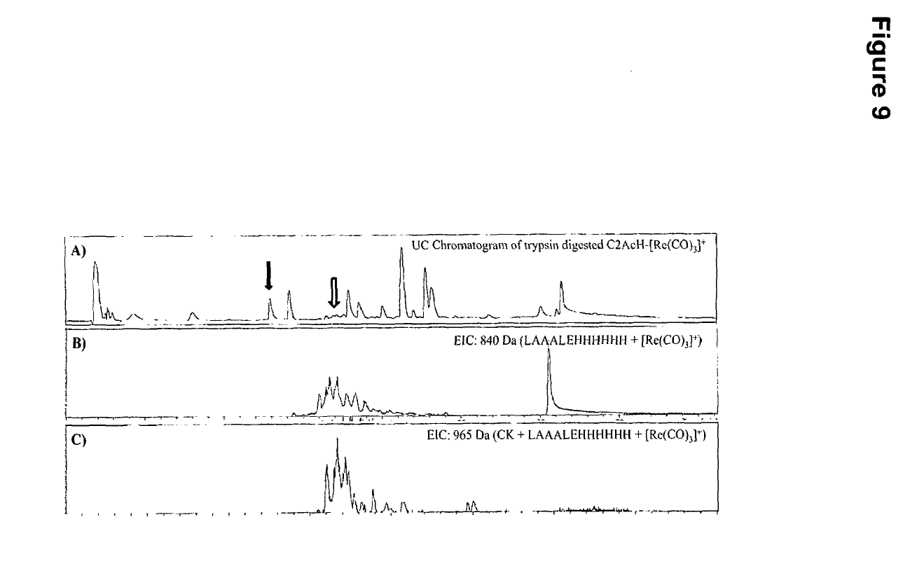

Upon further analysis of tryptic digests of both C2AcH-[Re(CO)₃]⁺ and C2AcH-A-[Re(CO)₃]⁺, multiple new peaks were noticed in the UV chromatogram upon digestion of the Re labeled C2AcH proteins (FIG. 8). This led to the possibility that [Re(CO)₃]⁺ bound to CKLAAALEHHHHHH or LAAALEHHHHHH could form various isomers. Multiple isomers are seen when looking at the extracted ion chromatogram corresponding to LAAALEHHHHHH-[Re(CO)₃]⁺, MW=840 (FIG. 9B), the extracted iron chromatogram for (CK+LAAALEHHHHHH-[Re(CO)₃]⁺), MW=965 (FIG. 9C).

TABLE 2

| Protein (M)/Peptide (P) | Expected (Da) | Found (Da) |
|---|---|---|
| C2AcH-^{187/185}Re(CO)₃ | 16787 | 16787 [M₁ + Re(CO)₃]⁺ |
| C2AcH-A-^{187/185}Re(CO)₃ | 16845 | 16845 [M₂ + ^{187/185}Re(CO)₃]⁺ |
| LAAALEHHHHHH | 705 | 705 [P + 2H]²⁺ |
| LAAALEHHHHHH-^{187/185}Re(CO)₃ | 840 | 840 [P₁ + ^{187/185}Re(CO)₃ + H]²⁺ |
| CKLAAALEHHHHHH | 820 | Not Observed |
| CₐKLAAALEHHHHHH | 849 | Not Observed |
| CKLAAALEHHHHHH-^{187/185}Re(CO) | 956 | 956 [P₂ + Re(CO)₃ + H]²⁺ |
| CₐKLAAALEHHHHHH-^{187/185}Re(CO)₃ | 985 | Not Observed |
| CK + LAAALEHHHHHH-^{187/185}Re(CO)₃ | 965 | 965 [CK + P₁ + Re(CO)₃ + H]²⁺ |
| or | | or |
| CKLAAALEHHHHHH-^{187/185}Re(CO)₃ + | | 965 [P₂ + Re(CO)₃ + H₂O + H]²⁺ |
| H₂O | | |
| CₐK + LAAALEHHHHHH-^{187/185}Re(CO) | 994 | Not Observed |
| or | | |
| CₐKLAAALEHHHHHH-^{187/185}Re(CO)₃ + | | |
| H₂O | | |

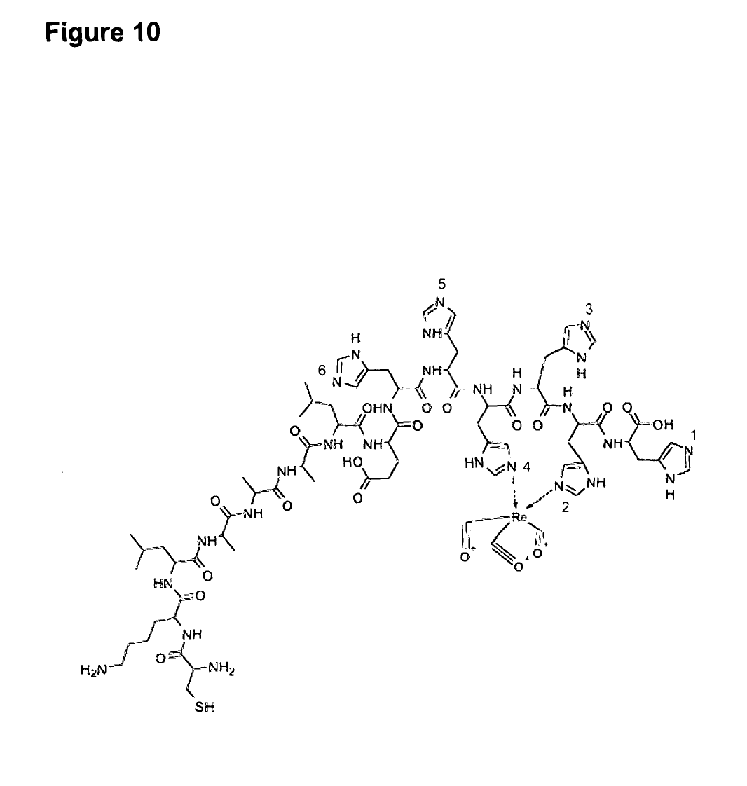

As the proposed binding of [Re(CO)₃]⁺ to His-tags involves two alternate histidines within the tag itself, there could be multiple coordinations of [Re(CO)₃]⁺ binding within the His-tag. A scheme of [Re(CO)₃]⁺ binding to CKLAAALEHHHHHH using ChemSketch (ACD/Labs, Toronto, Canada) is shown in FIG. 10.

The results of tryptic digest analysis and radiolabelling efficiencies suggest that the Cys and His-tag act synergistically to improve significantly the rate and efficiency of labelling with [^{99m}Tc(CO)₃]⁺ and [Re(CO)₃]⁺ compared to either protein with the His-tag alone, or the Cys alone, or neither.

The reaction of C2Ac with [Re(CO)₃]⁺ under these conditions did not give rise to a detectable rhenium conjugate and only unmodified C2Ac was detected by ES-MS. These results suggest that the Cys and His-tag act synergistically to improve significantly the rate and efficiency of labelling with [99 mTc(CO)₃]⁺ and [Re(CO)₃]⁺ compared to either protein with the His-tag alone, or the Cys alone, or neither.

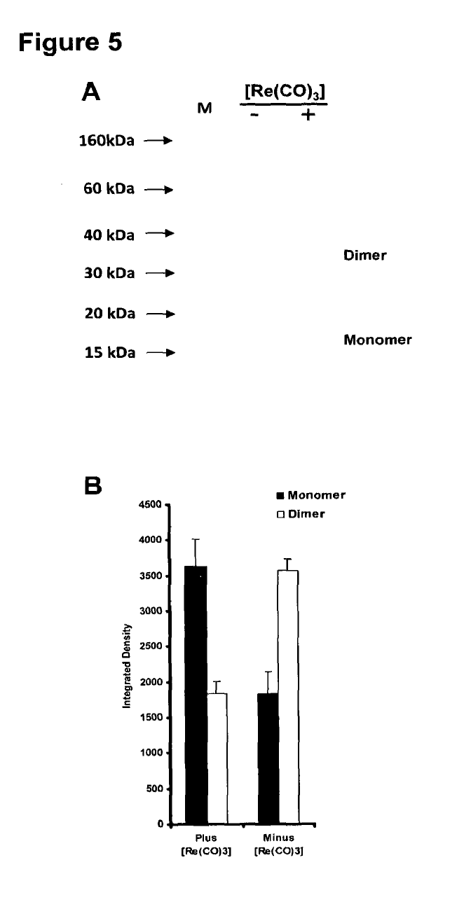

To investigate whether [Re(CO)₃]⁺ remained bound to the Cys, the dimerisation of the protein by aerial oxidation of the Cys thiol group was used as a probe (FIG. 5). Unmodified C2AcH undergoes extensive dimerisation (approx. 67% of protein) after exposure to air at room temperature at pH 8.0 for 16 h, as detected by non-reducing SDS/PAGE analysis. On the other hand, C2AcH that had been incubated at 37° C. with an ten fold excess of [(H₂O)₃Re(CO)₃]⁺ overnight, resulting in a preparation in which only the C2AcH-Re(CO)₃ conjugate, and no free C2AcH, could be detected by ES-MS, dimerisation was partially inhibited (30% of protein) but not completely blocked. This suggests the Re(CO)₂ fragment is engaged in binding with the Cys thiolate group, although not irreversibly, such that it can be made available for oxidation over time.

Site-Specifically ^{99m}Tc Labelled C2AcH Binds to Apoptotic Cells in a Calcium Dependent Manner

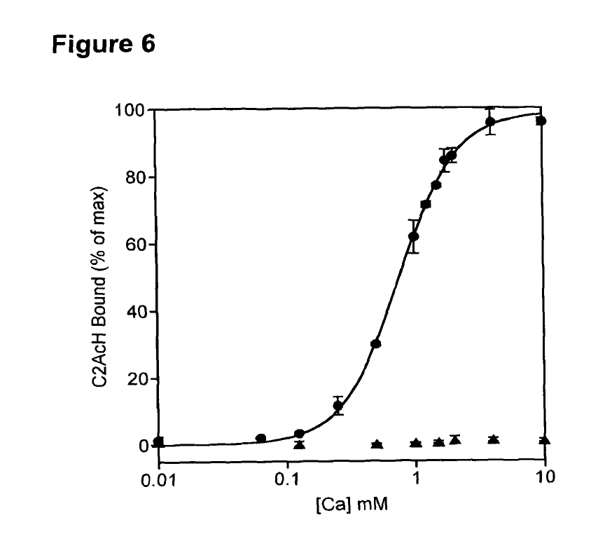

To determine whether or not radiolabeled C2AcH was still functional after radiolabelling, the protein was incubated with preserved red blood cells at varying concentrations of calcium from 0 to 10 mM (FIG. 6). Binding of ^{99m}Tc labeled C2AcH to red blood cells can be detected with as little as 0.25 mM CaCl₂ with maximal binding observed at 4 mM CaCl₂. After non-linear curve fitting (R=0.9996), the EC₅₀ of labelled C2AcH was calculated to be 0.76 mM of calcium, which is comparable to that observed for Annexin V (31) while for C2AcH-F the EC₅₀ was 1.03 mM (determined similarly, data not shown). To determine the comparative effect of non-site specific lysine modification, C2AcH was incubated with a twenty fold excess of the NHS derivative N-(benzyloxycarbonyloxy)succinimide and the resultant modified protein, C2AcH-B, was radiolabelled with ^{99m}Tc tricarbonyl with radiochemical purity >95% and subjected to the same PS-binding assay. No C2AcH-B calcium dependent binding to cells was observed at the highest concentration of Ca²⁺ of 10 mM (FIG. 6).

### Serum Stability of Radiolabelled C2AcH and C2AcH-F

The stabilities of C2AcH-[^{99m}Tc(CO)₃] and C2AcH-F-[^{99m}Tc(CO)₃] after incubation in PBS or serum over 18 h were determined (Table 3) using ITLC with gamma detection. C2AcH-[^{99m}Tc(CO)₃], C2AcH-F-[⁹⁹″Tc(CO)₃], [^{99m}TcO₄]⁻ and [(H₂O)₃^{99m}Tc(CO)₃] were analyzed beforehand in order to determine their migration on ITLC plates. For C2AcH-[^{99m}Tc(CO)₃], only one radioactive peak, with Rf=0, was observed for the duration of the stability study, both in PBS and serum. However, C2AcH-F-[^{99m}Tc(CO)₃] after 18 h in serum gave rise to a second minor peak with an Rf=1.0 with approximately 5% of the radioactivity, while the PBS control showed little degradation.

TABLE 3
Serum stability of technetium labelled
C2AcH and C2AcH-F proteins

Incubation Time (hrs)

| | | 0 | 3 | 6 | 18 |
|---|---|---|---|---|---|
| C2AcH | PBS Control | 99.54 | 99.34 | 98.81 | 99.22 |
| | Serum | 99.15 | 98.58 | 98.23 | 97.88 |
| C2AcH-F | PBS Control | 99.75 | 99.68 | 99.57 | 98.82 |
| | Serum | 99.38 | 97.24 | 96.31 | 94.39 |

Wild type mice (3) were injected i.v. with 2 MBq of C2AcH-[^{99m}Tc(CO)₃]⁺ and culled after 2 h. Organs were then weighed and counted for radioactivity using a gamma counter and presented as the percent injected dose per gram organ. High kidney uptake shows that C2AcH-[^{99m}Tc(CO)₃]⁺ is excreted through the urine. There was also moderate liver uptake.

### SPECT Imaging.

Wild type mice were injected i.v. into the tail vein with 20 MBq C2AcH-[^{99m}Tc(CO)₃]⁺. Under isofluorane anesthetics, mice were then imaged over time for up to 2 h. A CT scan was first performed for anatomical reference at 30 min. post-injection. SPECT images were then acquired at 30, 45, 60, 75, and 90 min. post-injection. Localization in the cortex of the kidney was seen as well as some uptake in the liver. Over time the amount of activity in the bladder increases due to renal excretion of C2AcH-[99 mTc(CO)₃]⁺.

### Discussion

The present invention discloses for the first time the use of a linker sequence to a polypeptide, where the linker sequence include the combination of a His-tag and a free cysteine residue as a tag for incorporation of imaging probes into recombinant proteins. Although it was designed for versatility, to allow the incorporation of both a radiolabel via the His-tag and other imaging probes via covalent modification of the Cys, an unexpected benefit was that the efficiency of labelling with [^{99m}Tc(CO)₃]⁺ dramatically increased, giving higher labelling yield and specific activity under mild conditions (37° C., 30 min.), than the His-tag alone (C2AcH-A, ˜83%, C2AcH-F, ˜60%) or Cys alone (C2Ac, ˜15%). Radiochemical yields as measured by ITLC always exceeded 96% at a concentration of 1 μg/μL of C2AcH. The remainder of the losses are accounted for by retention of labelled protein on the PD10 size exclusion column used for purification, rather than radiochemical impurity in the crude labelling preparation. Accordingly, this shows that the present invention provides a kit-based labelling method that may be used without need for post-labelling purification. Furthermore, the labelling methods described herein are improved over prior art approaches that require higher temperatures and/or longer incubation times to achieve adequate radiochemical yields with some His-tagged proteins (32).

The C2Ac construct without a His-tag achieved 15% radiochemical yield, which is comparable to reports in the literature (33). This raised the possibility that [Tc(CO)₃]⁺-labelling may not be fully site-specific. However, given the relative inefficiency of labelling in the absence of a His-tag alone, it seems unlikely that other potential binding sites in the native C2A molecule could compete significantly with the Cys-His-tag combination, for binding to [Tc(CO)₃]⁺ and it seems unlikely that non-specific labelling makes a significant contribution in the case of C2AcH. This is further supported by the mass spectrum of the [Re(CO)₃]⁺ adducts: despite the presence of a ten-fold excess of [(H₂O)₃Re(CO)₃]⁺ over protein, there is no evidence that more than one [Re(CO)₃]⁺ binds to C2AcH, and no evidence of any binding to C2Ac. This site specific labelling was confirmed by tryptic digest of C2AcH and C2AcH-A which indicated that only peptide sequences containing the His-Tag sequence contained the addition of one [Re(CO)₃]⁺. No other peptide from the tryptic digest contained the addition of [Re(CO)₃]⁺. It is also consistent with the excellent PS-binding affinity observed for C2AcH-[^{99m}Tc(CO)₃]. The calcium EC₅₀ data suggest that the PS-affinity of C2AcH-[^{99m}Tc(CO)₃] is slightly better than that of C2Ac-F. This is probably the results of the latter containing some non-site-specifically modified protein, as evidenced by the detection of some doubly modified protein in its mass spectrum.

The observation that conjugation with [Re(CO)₃]⁺ markedly reduces dimer formation by thiol oxidation in air suggests that the thiol group is protected. One possibility for such an observation is that the thiol binds to the metal. If this is the case, however, the Re—S interaction must be labile to some extent because aerial oxidation is merely slowed and not completely blocked (FIG. 5). In addition, if the thiol group plays a major part in maintaining the protein-metal bond one might expect C2Ac to undergo labelling more efficiently than is observed. An alternative model to account for the dimerization behaviour is that the proximity of the [M(CO)₃]⁺ (M=Tc, Re) group bound to the His-tag may induces a structural change or cause steric hindrance that inhibits disulfide bond formation without need to invoke a M-S bonding interaction. In this model, the role of the Cys thiol would be important in the initial formation of the protein-metal complex, but not in the final structure. This is consistent with the serum stability results, which show only a marginal (possibly insignificant) loss in stability by blocking the Cys thiol group with fluorescein-maleimide. Given the well-established role of cysteine thiol groups as powerful nucleophilic catalysts in many enzymes, it is conceivable that the thiol or thiolate group acts as a local catalyst by performing the initial nucleophilic attack on the positively charged rhenium tricarbonyl complex, to form an intermediate containing a M-S bond, followed by displacement of the thiolate from the metal coordination sphere by histidine.

The results we described suggest that the combination of Cys and His-tag is an excellent efficient sequence for labelling with radionuclide complexes, such as [Tc(CO)₃]⁺, with improved properties compared to the His-tag alone.

The PS-binding assay results with these proteins further highlight the value of a site-specific approach to labelling. The site-specifically labelled conjugates C2AcH-^{99m}Tc(CO)₃] and C2AcH-F bound to PS on RBCs in a calcium dependent manner. However, when non-specifically modified C2AcH-B, formed by incubation with an excess of N-(benzyloxycarbonyloxy)succinimide was radiolabelled it did not bind to PS on RBCs even at the highest concentrations of calcium.

In conclusion, the present invention provides a new class of radiopharmaceuticals that are especially useful for imaging cell death. Examples of these bioconjugates are based on C2A, the phosphatidylserine-binding domain of synaptotagmin I, although the approach could be extended to other polypeptides. It incorporates a novel [^{99m}Tc(CO)₃]⁺- and [Re(CO)₃]⁺-binding amino acid sequence that labels with excellent efficiency and site-specificity, with excellent serum stability, and is suitable for evaluation with other recombinant proteins for molecular imaging. The new site-specifically labelled C2AcH-[^{99m}Tc(CO)₃] has excellent affinity for phosphatidylserine, and may be employed in in vivo evaluation for cell death imaging in oncological, cardiovascular and graft rejection preclinical models.

While the foregoing invention has been described in some detail for purposes of clarity and understanding, it will be clear to one skilled in the art from a reading of this disclosure that various changes in form and detail may be made. For example, all the techniques and apparatus described above can be used in various combinations.

### REFERENCES

All publications, patents, patent applications, and/or other documents cited in this application are incorporated by reference in their entirety for all purposes to the same extent as if each individual publication, patent, patent application, and/or other document were individually indicated to be incorporated by reference for all purposes.
(1) Kerr, J. F., Wyllie, A. H., and Currie, A. R. (1972) Apoptosis: a basic biological phenomenon with wide-ranging implications in tissue kinetics. Br. J. Cancer 26, 239-57.
(2) Boersma, H. H., Kietselaer, B. L., Stolk, L. M., Bennaghmouch, A., Hofstra, L., Narula, J., Heidendal, G. A., and Reutelingsperger, C. P. (2005) Past, present, and future of annexin A5: from protein discovery to clinical applications. J. Nucl. Med. 46, 2035-50.
(3) Blankenberg, F. G., Katsikis, P. D., Tait, J. F., Davis, R. E., Naumovski, L., Ohtsuki, K., Kopiwoda, S., Abrams, M. J., Darkes, M., Robbins, R. C., Maecker, H. T., and Strauss, H. W. (1998) In vivo detection and imaging of phosphatidylserine expression during programmed cell death. Proc. Natl. Acad. Sci. U.S.A. 95, 6349-54.
(4) Fukuda, M., Kojima, T., and Mikoshiba, K. (1996) Phospholipid composition dependence of Ca2+-dependent phospholipid binding to the C2A domain of synaptotagmin IV. J. Biol. Chem. 271, 8430-4.
(5) Zhao, M., Zhu, X., Ji, S., Zhou, J., Ozker, K. S., Fang, W., Molthen, R. C., and Hellman, R. S. (2006) 99 mTc-labeled C2A domain of synaptotagmin I as a target-specific molecular probe for noninvasive imaging of acute myocardial infarction. J. Nucl. Med. 47, 1367-74.
(6) Hirt, U. A., and Leist, M. (2003) Rapid, noninflammatory and PS-dependent phagocytic clearance of necrotic cells. Cell Death Differ. 10, 1156-64.
(7) Blankenberg, F. G. (2008) Monitoring of treatment-induced apoptosis in oncology with PET and SPECT. Curr. Pharm. Des. 14, 2974-82.
(8) Giersing, B. K., Rae, M. T., CarballidoBrea, M., Williamson, R. A., and Blower, P. J. (2001) Synthesis and characterization of 111In-DTPA-N-TIMP-2: a radiopharmaceutical for imaging matrix metalloproteinase expression. Bioconjug. Chem. 12, 964-71.
(9) Tait, J. F., Smith, C., and Blankenberg, F. G. (2005) Structural requirements for in vivo detection of cell death with 99 mTc-annexin V. J. Nucl. Med. 46, 807-15.
(10) Zhu, X., Migrino, R. Q., Hellman, R. S., Brahmbhatt, T., and Zhao, M. (2008) Early uptake of 99 mTc-C2A in the acute phase of myocardial infarction as a prognostic indicator for follow-up cardiac dysfunction. Nucl. Med. Commun. 29, 764-9.
(11) Vriens, P. W., Blankenberg, F. G., Stoot, J. H., Ohtsuki, K., Berry, G. J., Tait, J. F., Strauss, H. W., and Robbins, R. C. (1998) The use of technetium Tc 99m annexin V for in vivo imaging of apoptosis during cardiac allograft rejection. J. Thorac. Cardiovasc. Surg. 116, 844-53.
(12) Ohtsuki, K., Akashi, K., Aoka, Y., Blankenberg, F. G., Kopiwoda, S., Tait, J. F., and Strauss, H. W. (1999) Technetium-99m HYNIC-annexin V: a potential radiopharmaceutical for the in-vivo detection of apoptosis. Eur. J. Nucl. Med. 26, 1251-8.
(13) Blankenberg, F. G., Vanderheyden, J. L., Strauss, H. W., and Tait, J. F. (2006) Radiolabeling of HYNIC-annexin V with technetium-99m for in vivo imaging of apoptosis. Nat Protoc 1, 108-10.
(14) Zhang, X., Rizo, J., and Sudhof, T. C. (1998) Mechanism of phospholipid binding by the C2A-domain of synaptotagmin I. Biochemistry 37, 12395-403.
(15) Sugita, S., and Sudhof, T. C. (2000) Specificity of Ca2+-dependent protein interactions mediated by the C2A domains of synaptotagmins. Biochemistry 39, 2940-9.
(16) Zhao, M., Beauregard, D. A., Loizou, L., Davletov, B., and Brindle, K. M. (2001) Non-invasive detection of apoptosis using magnetic resonance imaging and a targeted contrast agent. Nat. Med. 7, 1241-4.
(17) Jung, H. I., Kettunen, M. I., Davletov, B., and Brindle, K. M. (2004) Detection of apoptosis using the C2A domain of synaptotagmin I. Bioconjugate chemistry 15, 983-7.
(18) Krishnan, A. S., Neves, A. A., de Backer, M. M., Hu, D. E., Davletov, B., Kettunen, M. I., and Brindle, K. M. (2008) Detection of cell death in tumors by using MR imaging and a gadolinium-based targeted contrast agent. Radiology 246, 854-62.
(19) Wang, F., Fang, W., Zhao, M., Wang, Z., Ji, S., Li, Y., and Zheng, Y. (2008) Imaging paclitaxel (chemotherapy)-induced tumor apoptosis with 99 mTc C2A, a domain of synaptotagmin I: a preliminary study. Nucl. Med. Biol. 35, 359-64.
(20) Audi, S., Poellmann, M., Zhu, X., Li, Z., and Zhao, M. (2007) Quantitative analysis of [99 mTc]C2A-GST distribution in the area at risk after myocardial ischemia and reperfusion using a compartmental model. Nucl. Med. Biol. 34, 897-905.
(21) Tait, J. F., Smith, C., and Gibson, D. F. (2002) Development of annexin V mutants suitable for labeling with Tc(i)-carbonyl complex. Bioconjug. Chem. 13, 1119-23.
(22) Fonge, H., de Saint Hubert, M., Vunckx, K., Rattat, D., Nuyts, J., Bormans, G., Ni, Y., Reutelingsperger, C., and Verbruggen, A. (2008) Preliminary in vivo evaluation of a novel 99 mTc-labeled HYNIC-cys-annexin A5 as an apoptosis imaging agent. Bioorganic & medicinal chemistry letters 18, 3794-8.
(23) Waibel, R., Alberto, R., Willuda, J., Finnern, R., Schibli, R., Stichelberger, A., Egli, A., Abram, U., Mach, J. P., Pluckthun, A., and Schubiger, P. A. (1999) Stable one-step technetium-99m labeling of His-tagged recombinant proteins with a novel Tc(I)-carbonyl complex. Nat. Biotechnol. 17, 897-901.

1. A bioconjugate for use in imaging that comprises:
a polypeptide which is capable of interacting with a target of interest in a biological system; and
a linker sequence covalently bonded to the polypeptide, the linker sequence comprising (a) a free cysteine residue and (b) a polyhistidine sequence which is capable of site-specific labelling with a radionuclide for imaging the target of interest using the bioconjugate;
wherein both the free cysteine residue and the polyhistidine sequence are capable of simultaneously interacting with the radionuclide.

2. The bioconjugate of claim 1, wherein free cysteine and the polyhistidine sequences bind to a complex comprising the radionuclide.

3. The bioconjugate of claim 1, further comprising a complex comprising a radionuclide bound to the free cysteine residue and to the polyhistidine sequence.

4. The bioconjugate of claim 1, wherein the free cysteine residue is separated from the polyhistidine sequence by 5 to 10 amino acid residues.

5. The bioconjugate of claim 1, wherein the polyhistidine sequence has between 5 and 10 histidine residues.

6. The bioconjugate of claim 5, wherein the polyhistidine sequence has 5, 6 or 10 histidine residues.

7. The bioconjugate of claim 1, wherein the linker sequence is fused to the C-terminus of the protein domain.

8. The bioconjugate of claim 1, wherein linker sequence is represented by the general formula -Cys-Xₙ-His₅₋₁₀, where X is any amino acid residue and n is between 5 and 10.

9. The bioconjugate of claim 1, wherein the polypeptide is a protein domain or an antibody, or a fragment thereof.

10. The bioconjugate of claim 9, wherein the protein domain is a C2 domain of a phosphatidylserine binding protein.

11. The bioconjugate of claim 9, wherein the polypeptide is selected from the group consisting of, C2A domain of synaptotagmin I, an anti-CD33 antibody, TIMP-2, complement receptor 2 (CR2), an anti-CD169 antibody, an anti-CD68 antibody, an anti-CD64 antibody, and domains or fragments thereof.

12. The bioconjugate of claim 2, wherein the radionuclide is selected from the group consisting of isotopes of technetium, rhenium, copper, cobalt, gallium and indium.

13. The bioconjugate of claim 12, wherein the radionuclide is selected from the group consisting of Tc-99m, Re-186, Re-188, Co-57, Ga-67, In-111, Cu-64, Cu-60, Cu-61, Cu-62, Cu-67, Tc-94m, Ga-68 and Co-55.

14. The bioconjugate of claim 1, wherein the free cysteine is capable of site-specific linkage to a label.

15. The bioconjugate of claim 14, wherein the label is covalently linked to the linker sequence by a reaction with the free cysteine residue.

16. The bioconjugate of claim 14, wherein the free cysteine is covalently bonded to the label via a sulfhydryl-reactive maleimide, haloacetyl, pyridyldisulfide or vinyl sulfone group of the label reacting with the free thiol group of the cysteine residue.

17. The bioconjugate of a claim 14, wherein the label covalently bonded to is selected from the group consisting of a fluorescent label, a MRI contrast agent, a small molecule drug and a toxin.

18. The bioconjugate of claim 1, wherein the linker is expressed as a fusion with the polypeptide.

19. A kit for making a labelled bioconjugate for use in imaging employing a polypeptide which is capable of interacting with a target of interest in a biological system, the kit comprising:
(i) a linker sequence for covalently linking to the polypeptide, the linker sequence comprising (a) a free cysteine residue and (b) a polyhistidine sequence which is capable of site-specific labelling with a radionuclide for imaging the target of interest using the bioconjugate, wherein both the free cysteine residue and the polyhistidine sequence are capable of simultaneously interacting with the radionuclide, or a nucleic acid sequence encoding the linker sequence for ligation to a nucleic acid sequence encoding the polypeptide;
(ii) optionally, a reagent comprising the radionuclide for labelling the polyhistidine sequence;
(iii) optionally, a label for site-specific reaction with the free cysteine residue of the linker; and
(iv) optionally, one or more additional reagents for carrying out the radiolabelling or labelling reactions.

20. (canceled)

21. A method of making a bioconjugate for use in imaging, the method comprising:
(i) expressing a fusion protein of a polypeptide which is capable of interacting with a target of interest in a biological system and a linker sequence comprising (a) a free cysteine residue, wherein both the free cysteine residue and the polyhistidine sequence are capable of simultaneously interacting with the radionuclide, and (b) a polyhistidine sequence which is capable of site-specific labelling with a radionuclide for imaging the target of interest using the bioconjugate;
(ii) contacting the fusion protein with a complex comprising the radionuclide so that the complex binds to the polyhistidine sequence;
(iii) optionally reacting the free cysteine residue and the label so that the label covalently bonds to cysteine residue.

22. A method of imaging employing a bioconjugate that comprises:
a polypeptide which is capable of interacting with a target of interest in a biological system; and
a linker sequence covalently bonded to the polypeptide, the linker sequence comprising (a) a free cysteine residue and (b) a polyhistidine sequence which is capable of site-specific labelling with a radionuclide for imaging the target of interest using the bioconjugate, wherein both the free cysteine residue and the polyhistidine sequence are capable of simultaneously interacting with the radionuclide;
said method comprising the steps of:
(i) introducing the bioconjugate into the biological system comprising the target of interest.
(ii) detecting the radionuclide to image the target of interest in the biological system; and
(iii) optionally detecting the label.

23. The bioconjugate for use in a method of imaging of claim 22, wherein the method images cell death.

24. The bioconjugate for use in a method of imaging of claim 22, wherein the imaging is in vivo imaging.

25. The method of imaging of claim 22, wherein the method detects apoptosis.

26. The method of imaging of claim 23, wherein the imaging of cell death is for an oncological, cardiovascular or graft rejection application.

27. The method of imaging of claim 22, wherein the radionuclide is detected by SPECT or PET.

28. The method of imaging of claim 22, wherein the radionuclide and label are detected in a multi-modal imaging method.

29. (canceled)
